(19) United States
(12) Patent Application Publication
(10) Pub. No.: US 2009/0163449 A1
(43) Pub. Date: Jun. 25, 2009

(54) SULFO-POLYMER POWDER AND SULFO-POLYMER POWDER BLENDS WITH CARRIERS AND/OR ADDITIVES

(75) Inventor: Michael Fitzpatrick Wempe, Kingsport, TN (US)

Correspondence Address: BRETT L. NELSON;EASTMAN CHEMICAL COMPANY, 100 NORTH EASTMAN ROAD, KINGSPORT, TN, 37660-5075, US

(73) Assignee: Eastman Chemical Company, Kingsport, TN (US)

(21) Appl. No.: 12/262,986

(22) Filed: Oct. 31, 2008

Related U.S. Application Data
(60) Provisional application No. 61/015,365, filed on Dec. 20, 2007.

(51) Int. Cl. A61K 31/60; A61K 47/34

(52) U.S. Cl. 514/159; 514/772.3

(57) ABSTRACT

Sulfo-polyester powders and sulfo-polyester blend powders, the incorporation of carriers and/or actives, and methods of making the powders as well as dispersions employing these powders.

### CROSS-REFERENCE TO RELATED APPLICATIONS

This application claims priority to U.S. Provisional Application Ser. No. 61/015,365, filed Dec. 20, 2007 the disclosure of which is incorporated herein by reference in its entirety.

### BACKGROUND OF THE INVENTION

Sulfo-polyesters (such as Eastman AQ polymers) are high molecular weight amorphous polyesters commonly dispersed directly in water without the need to incorporate organic co-solvents, surfactants, or amines. Sulfo-polyesters differ chiefly by their chemical makeup (i.e. they are composed of 5-sodiosulfoisophthalic acid (5-SSIPA) and various combinations of other materials, for example: terephthalic acid (TPA), isophthalic acid (IPA), ethylene glycol (EG), diethylene glycol (DEG), 1,4-cyclohexanedimethanol (CHDM), and/or 1,4-cyclohexanedicarboxylic acid (1,4-CHDA). The temperature where a glassy polymer becomes rubbery on heating, and vice versa upon cooling, is known as the ‘glass transition temperature (T_{g}). Hence, the various sulfo-polyester polymers have different average T_{g} values.

Sulfopolyesters are solid to semi-solid polymers and require warm to hot water with sufficient mixing time to prepare concentrated dispersions. Moreover, different sulfo-polyester polymer dispersions may possess trace volatile components and potentially cause odor. Therefore methods that improve dissolution and/or lower volatile content may be advantageous.

### BRIEF SUMMARY OF THE INVENTION

The present invention addresses solutions to issues previously described and enables the preparation of sulfopolyester polymer powders and/or blends containing carriers (e.g. surfactants) and/or actives, and dispersions of sulfopolyester or sulfopolyester blends with and without carriers and/or actives, having reduced odor, using water and/or solvent mixtures with sufficient mixing.

An embodiment of the present invention concerns a composition comprising at least one sulfonated copolyester and at least one active agent, wherein said composition is a powder.

Yet another embodiment concerns a method of increasing the bioavailability of an active agent comprising administering the composition described in the previous paragraph to a subject (i.e. an organism).

Another embodiment concerns a method for producing a sulfonated copolyester powder containing an active agent comprising:

a) dispersing at least one sulfonated coployester and at least one active agent in a solvent, or solvent mixture, to form a dispersion; and

b) drying said dispersion to form a powder.

Another embodiment concerns a method for producing a sulfonated copolyester powder containing an active agent comprising:

a) dispersing at least one sulfonated copolyester in a solvent to form a dispersion; and

b) drying said dispersion to form a powder; and

c) mixing (grinding, milling, and the like) said sulfonated copolyester powder with at least one active agent.

Yet another embodiment concerns a cosmetic composition, comprising the compositions described above and a cosmetically acceptable carrier.

Another embodiment concerns a pharmaceutical or agricultural composition, comprising the compositions described above and a pharmaceutically or agriculturally acceptable carrier.

### BRIEF DESCRIPTION OF THE DRAWINGS

FIG. 1 shows a side-by-side comparison of a sulfopolyester polymer and the corresponding powder.

FIG. 2 shows the chemical structures for various compound examples incorporated with sulfopolyester polymer powders.

FIG. 3A shows the oral dose blood concentration-time data for itraconazole for two dosing groups; and

FIG. 3B shows the oral dose blood concentration-time data for hydroxy-itraconazole for two dosing groups.

### DETAILED DESCRIPTION

The present invention concerns sulfo-polyester powders and sulfo-polyester blend powders, the incorporation of carriers and/or actives, and methods of making the powders as well as dispersions employing these powders.

Sulfo-polyester powder(s) and sulfo-polyester powder blend(s) with and without carriers and/or actives are prepared by dispersing said sulfo-polyester powder(s) and sulfo-polyester powder blend(s) in a solvent or solvent mixture (e.g., water, water and alcohol mixtures, etc) at various temperatures. The exact solvent, or solvent mixture, and temperatures used will be a function of the application end usage and/or required dispersion percentage. End uses of said sulfo-polyester powder(s) and sulfo-polyester powder blend(s) include sun care/skin care applications (i.e. creams, lotions and sprays), perfume applications (i.e. flavors, fragrances, essential oils, etc), hairstyling applications (i.e. hair gel and hairspray), color makeup and hairstyling applications, drug-delivery applications, and adhesive removal such as re-pulping of paper and plastic and glass recycling.

As used throughout this application, the term “powder” shall mean particles in the range of 0.5-5000 μM. The science and technology of small particles is known as “micrometrics” (for a general review, see ‘Physical Pharmacy and Pharmaceutical Sciences’ Fifth Edition by Patrick J. Sinko, Lippincott Willams & Wilkins, 2006, ISBN: 0-7817-5027-X). The unit commonly used to describe particle size is the micrometer (μm). In general, optical microscopy may be used to measure particle-sizes of about 0.2 to about 100 μm; however, other techniques may also be used to determine approximate size ranges, such as sedimentation, coulter counter, airpermeability, sieving, etc.

Techniques such as sieving, according to methods of the U.S. Pharmacopeia, may be used to determine ‘powder fineness’, or other properties of the corresponding powders and/or powder blends; for example, particle size and size distribution (i.e. average particle size, particle-size distribution (frequency distribution curve), number and weight distributions, particle number), particle volume, particle shape and surface area, pore size, porosity, particle density, bulkiness, flow properties, etc. Because many powders have a tendency to contain a non-symmetric particle size distribution, it is common to plot the log-normal distribution; commonly, this method results in a linear relationship. Subsequently, the “geometric mean diameter” (d_{g}; the particle size equivalent to 50% on the probability scale) may be obtained from plotting the logarithm of the particle size against the cumulative percent frequency on a probability scale. Therefore, as used throughout this application, powders shall be classified into different particle size ranges, such as: ‘extremely fine powders’ (i.e. dusty powders; 0.5-50 μm), ‘fine powders’ (50-100 μm), ‘coarse powders’ (100-1000 μm), and ‘granular powders’ (1000-5000 μm).

In an embodiment of the present invention, the compositions are generally prepared by techniques such as lyophilization, spray-drying, jet milling, spray freeze-drying, fluidized-bed spray coating, supercritical fluid methods, etc. The different techniques are based on different mechanisms of droplet/particle formation and/or 1 5 drying (for a general review, see ‘Lyophilization of Biopharmaceuticals’ edited by Henry R. Costantino and Michael J. Pikal, AAPS Press, 2004, ISBN: 0-9711767-6-0).

The compositions are prepared by dispersing a sulfo-polyester or sulfo-polyester blend in a solvent, or solvent mixture (e.g. water, water and alcohol mixtures, etc), and optionally at least one active at various temperatures to form a dispersion. The dispersion can also include a carrier. The starting sulfo-polyester or sulfo-polyester blend can be in pellet form, or, for example, can be a sample that has been chipped or ground up. The exact solvent, or solvent mixture, and temperatures used will be a function of the application end usage and/or required dispersion percentage.

Typical starting dispersions contain about 35% to about 2% by weight sulfo-polyester or sulfo-polyester blend, or between about 25% to about 5% weight sulfo-polyester or sulfo-polyester blend, or between about 15% to about 7% by weight sulfo-polyester or sulfo-polyester blend.

A non-exhaustive list of suitable solvents, or solvent mixtures, includes: water, 30 methanol, ethanol, propanol, isopropyl alcohol, polyethylene glycols, propylene glycols, etc., and solvent mixtures thereof. Typically the solvent/sulfo-polyester or sulfo-polyester blend mixture is dispersed at a temperature of about 90° C. to about 20° C., about 70° C. to about 30° C., or about 60° C. to about 40° C. Typically, the mixtures are stirred at an elevated temperature until the sulfo-polyester or sulfo-polyester blend is dispersed which can require a time period of about 72 h to about 0.1 h, of about 48 h to about 1 h, of about 24 h to about 12 h, or about 6 h to about 2 h.

The powdered composition can then be formed via, for example, lyophilization wherein the dispersion is first frozen and then lyophilized. Alternatively, the powders may be formed via other methods (spray-drying, jet milling, spray freeze-drying, fluidized-bed spray coating, supercritical fluid methods, etc.).

The resulting powders are readily dispersible and show a significant reduction in residual volatile components; hence the sulfo-polyester powders are purer materials than the corresponding sulfopolyester. Furthermore, compared to the solid sulfo-polyester(s) on a weight to weight comparison, the corresponding solvent/sulfo-polyester powder or sulfo-polyester powder blends disperse at room temperature with rates which are statistically different from one another (for example, see the ‘Dispersion preparation from AQ powder example’); the powders have a much faster dissolution rate in a corresponding solvent, or solvent mixture, at room temperature. Various methods to evaluate dissolution are well known; for a general review, see ‘Physical Pharmacy and Pharmaceutical Sciences’ Fifth Edition by Patrick J. Sinko, Lippincott Willams & Wilkins, 2006, ISBN: 0-7817-5027-X. Therefore, one should readily appreciate that the application end usage and/or required dispersion percentage would dictate the exact solvent, or solvent mixture, and temperatures used to prepare dispersions from the powders. For example, a temperature of about 70° C. to about 10° C., about 50° C. to about 15° C., or about 35° C. to about 18° C. maybe used. Typically, the mixtures are stirred at room temperature, or elevated temperature, until the sulfo-polyester powder or sulfo-polyester powder blend becomes dispersed which can be a time period of about 12 h to about 1 min, of about 6 h to about 5 min, or of about 2 h to about 15 min.

As used throughout this application, the term “lipophilic compounds” shall mean compounds having solubility in water that is in the “sparingly soluble” range, or lower. Persons of ordinary skill in the art will understand that, for compounds that are “sparingly soluble in water,” the quantity of water needed to dissolve one gram of the compound will be in the range beginning at about 30 mL and ending at about 100 mL. Compounds having solubility lower than “sparingly soluble” in water will require greater volumes of water to dissolve the compounds.

As used throughout this application, the terms “Carrier”, “Carriers”, and “effectively solubilizing” a compound or having a “solubilizing effect” on such compound shall mean having the effect of increasing the solubility in water of the compound at least two-fold. Suitable carriers include water, alcohols, oils and the like, chosen for their ability to dissolve or disperse ingredients used in the treatment.

As used throughout this application, the term ‘“flavor” or “fragrance” or “essential oil” shall mean products derived from botanical sources and synthetically prepared from fossil fuels. A non-exhaustive list includes: Amyris oil, Anise oil, Basil oil, Bay oil, Beeswax, Benzoin resin, Bergamot oil, Birch oil, Birch tar oil, Black pepper oil, Bois de rose, Bran absolute, Cade oil, Camphor white oil, Canaga oil, Cardamom oil, Carrot seed oil, Cassia oil, Cassis bourgens absolute, Castor oil, Cedar leaf oil, Cedarwood oil, Cedrol, Cedryl acetate, Chamomile oil, Cinnamon bark oil, Cinnamon leaf oil, Citronella oil, Clary sage oil, Clove bud oil, Cognac oil, Copaiba balsam oil, Coriander oil, Commint oil, Costus oil, Dillweed oil, Elemi Resin, Eucalyptus oil, Fennel oil, Fenugreek absolute, Fir needle oil, Fir oil, Galbanum oil, Garlic oil, Genet absolute, Ginger oil, Grapefruit oil, Guaiac wood oil, Guaicwood acetate, Jasmin absolute Morocco, Juniperberry oil, Lavender oil 40/42% fleurs, Lemon oil, Lemongrass oil, Lemongrass oil, Lime oil, Litsea cubeba oil, Lovage oil, Mandarin oil, Marjoram oil, Methyl cedryl ether, Methyl cedryl ketone, Mimosa absolute Moroccan, Musk ketone, Myrrh oil, Nerolin bromeliad, Nerolin Yara Yara, Nutmeg oil, Oil of turpentine, Oilbanum oil, Onion oil, Opoponax oil, Orange oil, Orange terpenes, Origanum oil, Orris concrete, Osmanthus oil, Parsley oil, Patchouli oil, Peppermint oil, Petitgrain oil, Paraguay, Pimenta leaf oil, Rose absolute, Moroccan, Rose crystals, Rose oil, Rosemary oil, Sage oil, Sandalwood oil, Sassafras oil, Spearmint oil, Spike lavender oil, Styrax, Tarragon oil, Tea tree oil, Terpineol, Thyme oil, Tolu balsam, Trivertal, Vetiver acetate, Violet leaf absolute, Wintergreen oil, Ylang-ylang oil, (−)-Ambroxide, (−)-Bornyl isovalerate, (+)-Arabinogalactan, 2′-Aminoacetophenone, 2-Acetyl-1-methylpyrrole, 2-Acetyl-3,5(6)-dimethylpyrazine, 2-Acetyl-3-ethylpyrazine, 2-Acetyl-3-methylpyrazine, 2-Acetyl-5-methylfuran, 2-Acetylpyrazine, 2-Acetylpyridine, 2-Acetylthiazole, 2-Acetylthiophene, 2-Butanol, 2-Butanone, 2-sec-Butylcyclohexanone, 3-Acetyl-2,5-dimethylthiophene, 3-Acetyl-2,5-dimethylfuran, 3-Acetylpyridine, 3-Butylidenephthalide, 4-(p-Acetoxyphenyl)-2-butanone, 4-Acetoxy-2,5-dimethyl-3(2H)furanone, 4-Allyl-1,2-dimethoxybenzene, 4-Allyl-2,6-dimethoxyphenol, 4-Allylanisole, 4-tert-Butylcyclohexyl acetate, 4-tert-Butylphenol , 5α-Androst-16-en-3-one, 5α-Androst-16-en-3α-ol, 6-Acetoxydihydrotheaspirane, 6-Acetyl-1,1,2,4,4,7-hexamethyltetralin, 6-Amyl-α-pyrone, Acetal, Acetaldehyde, Acetanisole, Acetoacetanilide, Acetophenone, Acetovanillone, Adipic acid, Allyl 2-ethylbutyrate, Allyl 2-furoate, Allyl anthranilate, Allyl butyrate, Allyl cinnamate, Allyl cyclohexanepropionate, Allyl disulfide, Allyl heptanoate, Allyl hexanoate, Allyl isoamyl glycolate, Allyl isothiocyanate, Allyl isovalerate, Allyl octanoate, Allyl phenoxyacetate, Allyl phenylacetate, Allyl propionate, Allyl thiopropionate, Allyl tiglate, Allyl-α-ionone, Amyl 2-furoate, Amyl acetate, Amyl alcohol, Amyl butyrate, Amyl formate, Amyl hexanoate, Amyl octanoate, mixture of isomers, Anisyl acetate, Anisyl alcohol, Anisyl alcohol natural, Anisyl formate, Anisyl phenylacetate, Anisyl propionate, Benzaldehyde propylene glycol acetal, Benzaldehyde, Benzenethiol, Benzoic acid, Benzophenone, Benzothiazole, Benzyl acetate, Benzyl Acetoacetate, Benzyl alcohol, Benzyl benzoate, Benzyl butyrate, Benzyl cinnamate, Benzyl isovalerate, Benzyl mercaptan, Benzyl phenylacetate, Benzyl propionate, Benzyl propionate natural, Benzyl salicylate, Benzylideneacetone, Biphenyl, Bis(2-ethylhexyl)Adipate, Bis(methylthio)methane, Bornyl acetate, Bornyl valerate, Butan-3-one-2-yl butanoate, Butyl 10-undecenoate, Butyl 2-methylbutyrate, Butyl 4-hydroxybenzoate, Butyl acetate, Butyl alcohol, Butyl anthranilate, Butyl benzoate, Butyl butyrate, Butyl butyryllactate, Butyl formate, Butyl heptanoate, Butyl hexanoate, Butyl isobutyrate, Butyl isovalerate, Butyl laurate, Butyl levulinate, Butyl phenylacetate, Butyl propionate, Butyl salicylate, Butyl valerate, Butylamine, Butyraldehyde, Butyric acid, Butyrophenone, N-Amyl octanoate, α-Amylcinnamaldehyde dimethyl acetal, α-Amylcinnamaldehyde, α-Amylcinnamyl alcohol, α-Angelica lactone, m-Anisaldehyde, m-Anisic acid, o-tert-Butylcyclohexyl acetate, p-Anisaldehyde, p-Anisic acid, trans-Aconitic acid, trans-Anethole, (−)-Carvyl acetate, (−)-Carvyl propionate, (−)-Dihydrocarvyl acetate, (±)-Camphor, (±)-Citronellal, (+)-Camphene, (+)-γ-Decalactone, (S)-(−)-β-Citronellol, 1,1-Dimethoxyethane, 1,2-Dimethoxybenzene, 1,3-Dihydroxyacetone dimer, 1,3-Dimethoxybenzene, 1,3-Diphenyl-2-propanone, 1,4-Dimethoxybenzene, 1,4-Dithiane, 1-Decanol, 1-Decen-3-ol, 2′,4′-Dimethylacetophenone, 2,2′-(Dithiodimethylene)difuran, 2,2-Dimethyl-5-(1-methylpropen-1-yl)tetrahydrofuran, 2,3-Diethyl-5-methylpyrazine, 2,3-Diethylpyrazine, 2,3-Dimethylbenzofuran, 2,3-Dimethylpyrazine, 2,3-Dimethylquinoxaline, 2,4-Dihydroxybenzoic acid, 2,4-Dimethyl-3-cyclohexenecarboxaldehyde, 2,4-Dimethyl-5-acetylthiazole, 2,4-Dimethylanisole, 2,4-Dimethylbenzaldehyde, 2,4-Dimethylthiazole, 2,5-Dimethyl-1,4-dithiane, 2,5-Dimethyl-4-methoxy-3(2H)-furanone, 2,5-Dimethylpyrazine, 2,5-Dimethylthiophene, 2,6-Diisopropylphenol, 2,6-Dimethoxyphenol, 2,6-Dimethyl-4-heptanol, 2,6-Dimethyl-4-heptanone, 2,6-Dimethyl-5-heptenal, 2,6-Dimethyl-7-octen-2-ol, 2,6-Dimethylbenzenethiol, 2,6-Dimethylpyrazine, 2,6-Dimethylpyridine, 2-Decanone, 3′,4′-Dimethoxyacetophenone, 3,4-Dimethoxybenzaldehyde, 3,4-Dimethyl-1,2-cyclopentadione, 3,5-Dimethyl-1,2-cyclopentadione, 3,7-Dimethyl-1-octanol, 3,7-Dimethyl-2,6-octadienenitrile, 3,7-Dimethyl-6-octenoic acid, 3-Carene, 3-Decanone, 3-Decen-2-one, 4,5-Dihydro-3(2H)-thiophenone, 4,5-Dimethyl-3-hydroxy-2,5-dihydrofuran-2-one, 4,5-Dimethylthiazole, 4-Carvomenthenol, 6,10-Dimethyl-5,9-undecadien-2-one, D-Camphor, D-Carvone, D-Dihydrocarvone, L-Carveol, Caffeine, Carvacrol, Carvacryl ethyl ether, Caryophyllene oxide, Cedrol, Cedryl acetate, Cinnamaldehyde, Cinnamyl acetate, Cinnamyl alcohol, Cinnamyl cinnamate, Cinnamyl formate, Cinnamyl isobutyrate, Citral diethyl acetal, Citral dimethyl acetal, Citral, Citric acid, Citronellol, Citronellyl acetate, Citronellyl formate, Citronellyl isobutyrate, Citronellyl propionate, Citronellyl propionate, Citronellyl tiglate, Cuminaldehyde,Cyclohexaneacetic acid, Cyclohexanecarboxylic acid, Cyclohexaneethyl acetate, Cyclohexanone, Cyclohexyl acetate, Cyclohexyl butyrate, Cyclohexyl isovalerate, Cyclohexyl propionate, Cyclopentanethiol, Cyclopentanone, Decahydro-2-naphthol, Decanal dimethyl acetal, Decanal, Decanoic acid, Decyl butyrate, Decyl propionate, Di(ethylene glycol) ethyl ether, Diacetin, Diethyl malate, Diethyl malonate, Diethyl phthalate, Diethyl sebacate, Diethyl succinate, Dihydro-β-ionone, Dihydrocarveol, Dihydrocoumarin, Dihydrojasmone, Dimethyl anthranilate, Dimethyl sulfide, Dimethyl trisulfide, Diphenyl ether, Disodium succinate, Dodecyl aldehyde, α,α-Dimethylphenethyl acetate, α,α-Dimethylphenethyl butyrate, β-Caryophyllene, β-Cyclocitral, cis-4-Decenal, δ-Decalactone, δ-Decalactone, δ-Dodecalactone, ε-Decalactone, γ-Decalactone, γ-Dodecalactone, m-Cresol, n-Decyl acetate, o-Cresol , p,α-Dimethylstyrene, p-Cresol, p-Cymene, trans-2-Decenal, trans-2-Dodecenal, trans-Cinnamaldehyde, trans-Cinnamic acid, trans-Cinnamyl butyrate, trans-Cinnamyl isovalerate, trans-Cinnamyl propionate, 1,6-Hexanedithiol, 1-Hexadecanol, 1-Hexanethiol, 1-Hexen-3-ol, 2′-Hydroxyacetophenone, 2,3-Heptanedione, 2,3-Hexanedione, 2-Heptanol, 2-Heptanone, 2-Heptylfuran, 2-Hydroxy-4-methylbenzaldehyde, 3,4-Hexanedione, 3-Heptanol, 3-Heptanone, 3-Hexanol, 3-Hexanone, 4-(4-Hydroxyphenyl)-2-butanone, 4-Heptanone, 4-Hexen-1-ol, 4-Hexen-3-one, 4-Hydroxy-2,5-dimethyl-3(2H)-furanone, 4-Hydroxy-3-methoxybenzyl alcohol, 4-Hydroxybenzaldehyde, 4-Hydroxybenzoic acid, 4-Hydroxybenzyl alcohol, 4-Hydroxybutanoic acid lactone, 5-(Hydroxymethyl)furfural, 5-Hydroxy-4-octanone, Geraniol, Geranyl acetate, Geranyl benzoate, Geranyl butyrate, Geranyl formate, Geranyl isovalerate, Geranyl phenylacetate, Geranyl propionate, Guaiacol, Guaiacyl phenylacetate, Guaicwood acetate, Heptaldehyde, Heptanal, Heptanoic acid, Heptyl acetate, Heptyl alcohol, Heptyl butyrate, Heptyl formate, Heptyl isobutyrate, Hexanal, Hexanoic acid, Hexyl 2-methylbutanoate, Hexyl 3-methylbutanoate, Hexyl acetate, Hexyl alcohol, Hexyl benzoate, Hexyl butyrate, Hexyl formate, Hexyl hexanoate, Hexyl isobutyrate, Hexyl octanoate, Hexyl phenylacetate, Hexyl propionate, Hexyl salicylate, Hexyl tiglate, Hexyl trans-2-butenoate, Hydrocinnamaldehyde, Hydroxycitronellal dimethyl acetal, Hydroxycitronellal, α-Hexylcinnamaldehyde, cis-2-Hexen-1-ol, cis-3-Hepten-1-ol, cis-3-Hexen-1-ol, cis-3-Hexen-1-ol, cis-3-Hexenyl 2-methylbutanoate, cis-3-Hexenyl 3-methylbutanoate, cis-3-Hexenyl acetate, cis-3-Hexenyl benzoate, cis-3-Hexenyl butyrate, cis-3-Hexenyl cis-3-hexenoate, cis-3-Hexenyl crotonate, cis-3-Hexenyl formate, cis-3-Hexenyl hexanoate , cis-3-Hexenyl isobutyrate, cis-3-Hexenyl lactate, cis-3-Hexenyl phenylacetate, cis-3-Hexenyl propionate, cis-3-Hexenyl salicylate, cis-3-Hexenyl tiglate, cis-4-Heptenal, δ-Hexalactone, γ-Heptalactone, γ-Hexalactone, ω-6-Hexadecenlactone, trans, trans-2,4-Heptandienal, trans,trans-2,4-Hexadien-1-ol, trans,trans-2,4-Hexadienal, trans-2-Heptenal, trans-2-Hexen-1-al , trans-2-Hexen-1-ol, trans-2-Hexenoic acid, trans-2-Hexenyl acetate, trans-2-Hexenyl butyrate, trans-3-Hexen-1-ol, trans-3-Hexenoic acid, (−)-Isopulegol, (R)-(+)-Limonene, (S)-(−)-Limonene, 2-Isobutyl-3-methoxypyrazine, 2-Isobutyl-3-methylpyrazine, 2-Isobutyl-4-methyl-1,3-dioxolane, 2-Isobutylthiazole, 2-Isopropyl-4-methylthiazole, 2-Isopropyl-5-methyl-2-hexenal, 2-Isopropylphenol, 4-Isopropylbenzyl alcohol, 4-Isopropylphenol, D-Isoascorbic acid, L-Linalool, Indole, Isoamyl 3-(2-furan)propionate, Isoamyl acetate, Isoamyl alcohol, Isoamyl benzoate, Isoamyl butyrate, Isoamyl cinnamate, Isoamyl formate, Isoamyl hexanoate, Isoamyl isobutyrate, Isoamyl isovalerate, Isoamyl laurate, Isoamyl nonanoate, Isoamyl octanoate, Isoamyl propionate, Isoamyl pyruvate, Isoamyl salicylate, Isoamyl tiglate, Isoborneol, Isobornyl acetate, Isobornyl propionate, Isobutyl acetate, Isobutyl alcohol, Isobutyl angelate, Isobutyl benzoate, Isobutyl butyrate, Isobutyl cinnamate, Isobutyl formate, Isobutyl hexanoate, Isobutyl isobutyrate, Isobutyl phenylacetate, Isobutyl propionate, Isobutyl salicylate, Isobutyl tiglate, Isobutyl trans-2-butenoate, Isobutyraldehyde, Isobutyric acid, Isoeugenol, Isoeugenyl acetate, Isoeugenyl phenylacetate, Isopentylamine, Isophorone, Isophytol, Isopropyl 2-methylbutyrate, Isopropyl acetate, Isopropyl alcohol, Isopropyl butyrate, Isopropyl cinnamate, Isopropyl disulfide, Isopropyl myristate, Isopropyl palmitate, Isopropyl phenylacetate, Isopropyl tiglate, Isovaleraldehyde, Isovaleric acid, Lauric acid, Lauryl acetate, Lauryl alcohol, Levulinic acid, Linalool, Linalyl acetate, Linalyl benzoate, Linalyl butyrate, Linalyl formate, Linalyl isovalerate, Linalyl propionate, Linoleic acid, α-Ionone, α-Isobutylphenethyl alcohol, cis-Jasmone, trans-Isoeugenyl benzyl ether, (±)-2-Methylbutyric acid, (±)-3-Methylvaleric acid, (±)-4-Methyloctanoic acid, (1R)-(−)-Myrtenal, (1R)-(−)-Nopyl acetate, (1S,2S,5R)-(+)-Neomenthol, (Methylthio)methylpyrazine, 1,3-Nonanediol acetate, 1,9-Nonanedithiol, 1-(p-Methoxyphenyl)-2-propanone, 1-Methyl-1,4-cyclohexadiene, 1-Methyl-3-methoxy-4-isopropylbenzene, 1-Methylnaphthalene, 1-Methylpiperidine, 1-Methylpyrrole, 2-(1-Methylpropyl)thiazole, 2-(Methylthio)ethanol, 2-,3-,10-Mercaptopinane, 2-Mercapto-3-butanol, 2-Mercaptopropionic acid, 2-Methoxy-3(5 or 6)-isopropylpyrazine, 2-Methoxy-3-(1-methylpropyl)pyrazine, 2-Methoxy-3-methylpyrazine, 2-Methoxy-4-methylphenol, 2-Methoxy-4-propylphenol, 2-Methoxy-4-vinylphenol, 2-Methoxyphenyl acetate, 2-Methoxypyrazine, 2-Methyl-1-butanethiol, 2-Methyl-1-butanol, 2-Methyl-1-propanethiol, 2-Methyl-2-pentenal, 2-Methyl-2-pentenoic acid, 2-Methyl-2-thiazoline, 2-Methyl-3(5 or 6)-ethoxypyrazine, 2-Methyl-3-(3,4-methylenedioxyphenyl)-propanal, 2-Methyl-3-(p-isopropylphenyl)Propionaldehyde, 2-Methyl-3-buten-2-ol, 2-Methyl-3-furanthiol, 2-Methyl-3-heptanone, 2-Methyl-3-methylthiofuran, 2-Methyl-4-pentenoic acid, 2-Methyl-4-propyl-1,3-oxathiane, 2-Methylanisole, 2-Methylbutyl 2-methylbutyrate, 2-Methylbutyl acetate, 2-Methylbutyl isovalerate, 2-Methylbutyraldehyde, 2-Methylbutyric acid, 2-Methylcyclohexanone, 2-Methylheptanoic acid, 2-Methylhexanoic acid, 2-Methylpentanal, 2-Methylpentanoic acid, 2-Methylpyrazine, 2-Methylquinoxaline, 2-Methyltetrahydro-3-furanone, 2-Methyltetrahydrothiophen-3-one, 2-Methylundecanal, 2-Nonanol, 2-Nonanone, 3-(5-Methyl-2-furyl)butanal, 3-(Methylthio)-1-hexanol, 3-(Methylthio)-1-propanol, 3-(Methylthio)butanal, 3-(Methylthio)hexyl acetate, 3-(Methylthio)Propionaldehyde, 3-(Methylthio)propyl isothiocyanate, 3-Mercapto-2-butanone, 3-Methyl-1,2-cyclohexanedione, 3-Methyl-1-butanethiol, 3-Methyl-1-pentanol, 3-Methyl-2-butanethiol, 3-Methyl-2-buten-1-ol, 3-Methyl-2-cyclohexenone, 3-Methyl-3-buten-1-ol, 3-Methyl-3-pentanol, 3-Methylbutyl 2-methylbutanoate, 3-Methylcrotonic acid, 3-Methylcyclohexanone, 3-Nonanone, 4′-Methylacetophenone, 4-(3,4-Methylenedioxyphenyl)-2-butanone, 4-(4-Methoxyphenyl)-2-butanone, 4-(Methylthio)butanol, 4-Methyl-1-phenyl-2-pentanone, 4-Methyl-2,6-dimethoxyphenol, 4-Methyl-2-pentanone, 4-Methyl-2-phenyl-2-pentenal, 4-Methyl-3-penten-2-one, 4-Methyl-5-thiazoleethanol acetate, 4-Methyl-5-thiazoleethanol, 4-Methyl-5-vinylthiazole, 4-Methylanisole, 4-Methylbiphenyl, 4-Methylcyclohexanone, 4-Methylnonanoic acid, 4-Methylquinoline, 4-Methylthiazole, 4-Methylthio-2-butanone, 4-Methylthio-4-methyl-2-pentanone, 4-Methylvaleric acid, 5-Methyl-2-thiophenecarboxaldehyde, 5-Methyl-2-hepten-4-one, 5-Methyl-2-phenyl-2-hexenal, 5-Methylfurfural, 5-Methylquinoxaline, 5H-5-Methyl-6,7-dihydrocyclopenta[b]pyrazine, 6-Methyl-5-hepten-2-ol , 6-Methyl-5-hepten-2-one, 6-Methylcoumarin, 6-Methylquinoline, 7-Methoxycoumarin, DL-3-Methyl-2-butanol, DL-Menthol, DL-Menthyl acetate, L-Menthol, L-Menthone, L-Menthyl acetate, Maltol, Maltyl isobutyrate, Menthalactone, Menthyl isovalerate, Methyl(methylthio)acetate, Methyl(p-tolyloxy)acetate, Methyl 2-furoate, Methyl 2-methoxybenzoate, Methyl 2-methylbutyrate, Methyl 2-methylpentanoate, Methyl 2-nonynoate, Methyl 2-octynoate, Methyl 2-pyrrolyl ketone, Methyl 2-thiofuroate, Methyl 3-(methylthio)propionate, Methyl 3-hydroxyhexanoate, Methyl 3-nonenoate, Methyl 4-hydroxybenzoate, Methyl 4-methylvalerate, Methyl acetate, Methyl anthranilate, Methyl atratate, Methyl β-naphthyl ketone, Methyl benzoate, Methyl butyrate, Methyl cedryl ether, Methyl cedryl ketone, Methyl cinnamate, Methyl cyclohexanecarboxylate, Methyl cyclopentenolone, Methyl decanoate, Methyl dihydrojasmonate, Methyl heptanoate, Methyl hexanoate, Methyl isobutyrate, Methyl isoeugenol, Methyl isovalerate, Methyl jasmonate, Methyl laurate, Methyl myristate, Methyl nicotinate, Methyl nonanoate, Methyl octanoate, Methyl p-anisate, Methyl p-tert-butylphenylacetate, Methyl palmitate, Methyl phenyl sulfide, Methyl phenylacetate, Methyl propionate, Methyl propyl disulfide, Methyl salicylate, Methyl stearate, Methyl thiobutyrate, Methyl tiglate, Methyl trans-2-nonenoate, Methyl trans-2-octenoate, Methyl trans-3-hexenoate, Methyl trans-cinnamate, Methyl valerate K, Methyl vanillate, Musk ketone, Myrcene, Myristic acid, Myrtenol, Myrtenyl acetate, Neohesperidin dihydrochalcone, Nerol, Neryl acetate, Neryl butyrate, Neryl isobutyrate, Neryl isovalerate, Nonanal, Nonanoic acid, Nonyl acetate, Nonyl alcohol, α-Methylbenzyl acetate, α-Methylbenzyl alcohol, α-Methylbenzyl butyrate, α-Methylbenzyl propionate, α-Methylcinnamaldehyde , cis-2-Nonen-1-ol, cis-6-Nonen-1-ol, cis-6-Nonenal, δ-Nonalactone, γ-Nonalactone, o-Methoxycinnamaldehyde, p-Mentha-8-thiol-3-one, trans, trans-2,4-Nonadienal, trans, trans-2,6-Nonadienal, trans-2,cis-6-Nonadien-1-ol, trans-2,cis-6-Nonadienal, trans-2,cis-6-Nonadienyl acetate, trans-2-Methyl-2-butenal, trans-2-Methyl-2-butenoic acid, trans-2-Nonen-1-ol, trans-2-Nonenal, trans-p-Methoxycinnamaldehyde, (−)-α-Pinene, (−)-β-Pinene, (±)-2-Pentanol, (1R)-(+)-α-Pinene, (R)-(+)-Pulegone, (S)-(−)-Perillaldehyde, (S)-(−)-Perillyl alcohol, 1,2-Propanediol, 1,3-Propanedithiol, 1,5-Pentanedithiol, 1,8-Octanedithiol, 1-Octanol, 1-Octen-3-ol , 1-Octen-3-yl acetate, 1-Octen-3-yl butyrate, 1-Penten-3-ol, 1-Phenyl-1,2-propanedione, 1-Phenyl-1-propanol, 1-Phenyl-3-methyl-3-pentanol, 1-Propanol, 2,3-Pentanedione, 2,4-Octadienal, 2-(3-Phenylpropyl)pyridine, 2-Octanol, 2-Octanone, 2-Oxobutyric acid, 2-Pentadecanone, 2-Pentanone, 2-Pentyl butyrate, 2-Pentylfuran, 2-Pentylpyridine, 2-Phenethylamine, 2-Phenoxyethyl isobutyrate, 2-Phenyl-1-propanol, 2-Phenyl-2-butenal, 2-Phenylethyl isothiocyanate, 2-Phenylphenol, 2-Phenylpropionaldehyde dimethyl acetal, 2-Phenylpropionaldehyde, 2-Phenylpropyl isobutyrate, 2-Propanethiol, 2-Propyl-4-methyl-1,3-dioxolane, 2-Propylphenol, 3-Octanol, 3-Octanone, 3-Octyl acetate, 3-Pentanone, 3-Phenyl propyl propionate, 3-Phenyl-1-propanol, 3-Phenylpropionic acid, 3-Phenylpropyl acetate, 3-Phenylpropyl isobutyrate, 3-Phenylpropyl isovalerate, 3-Propylidenephthalide, 4-Oxoisophorone, 4-Pentenoic acid, 4-Propylphenol, 5-Phenyl-1-pentanol, Octanal, Octanoic acid, Octyl acetate, Octyl butyrate, Octyl formate, Octyl isobutyrate, Octyl isovalerate, Octyl octanoate, Oleic acid, Palmitic acid, Phenethyl 2-furoate, Phenethyl 2-methylbutyrate, Phenethyl acetate, Phenethyl alcohol, Phenethyl anthranilate, Phenethyl benzoate, Phenethyl butyrate, Phenethyl cinnamate, Phenethyl formate, Phenethyl hexanoate, Phenethyl isobutyrate, Phenethyl isovalerate, Phenethyl octanoate, Phenethyl phenylacetate, Phenethyl propionate, Phenethyl salicylate, Phenethyl tiglate, Phenol, Phenoxyacetic acid, Phenoxyethyl propionate, Phenyl acetate, Phenyl salicylate, Phenylacetaldehyde dimethyl acetal, Phenylacetaldehyde, Phenylacetic acid, Phenylethyl mercaptan, Phytol, Piperidine, Piperine, Piperonal, Piperonyl acetate, Piperonyl isobutyrate, Polysorbate 20, Polysorbate 60, Polysorbate 80, Propenyl guaethol, Propionaldehyde, Propionic acid, Propiophenone, Propyl acetate, Propyl butyrate, Propyl disulfide, Propyl formate, Propyl gallate, Propyl heptanoate, Propyl hexanoate, Propyl isobutyrate, Propyl mercaptan, Propyl phenylacetate, Propyl propionate, Pyrazine, Pyrazineethanethiol, Pyridine, Pyroligneous acid, Pyrrole, Pyrrolidine, Pyruvic acid, α-Phellandrene, cis-2-Penten-1-ol, cis-5-Octen-1-ol, γ-Octalactone, ω-Pentadecalactone, p-Propyl anisole, trans-2-Octen-1-ol , trans-2-Octenal, trans-2-Pentenal, trans-3-Octen-2-one, (−)-α-Terpineol , (±)-Theaspirane, (±)-γ-Valerolactone, (1S)-(−)-Verbenone, 1,2,3,4-Tetrahydroquinoline, 1,2,6-Trihydroxyhexane, 1,3,5-Undecatriene, 10-Undecenal, 2,2′-(Thiodimethylene)difuran, 2,2,6-Trimethylcyclohexanone, 2,3,5,6-Tetramethylpyrazine, 2,3,5-Trimethylpyrazine, 2,3,6-Trimethylphenol, Xylenol, 2,6,6-Trimethyl-1-cyclohexene-1-acetaldehyde, 2-Thienyl disulfide, 2-Thiophenethiol, 2-Tridecanone, 2-Undecanone, 3,5,5-Trimethyl-1-hexanol, 3,5,5-Trimethylhexanal, 3-[5,5,6-Trimethylbicyclo[2.2.1]hept-2-yl]cyclohexan-1-ol, 4,5,6,7-Tetrahydro-3,6-dimethylbenzofuran, 5,6,7,8-Tetrahydroquinoxaline, 6-Undecanone, D-Sorbitol, 30 Glyceryl tributyrate, Quinoline, Resorcinol, Rhodinyl acetate, Rum Ether, Safranal, Salicylaldehyde, Salicylic acid, Skatole, Sorbic acid, Sorbitan monostearate, Stearic acid, Sucrose acetate isobutyrate, Sucrose octaacetate, Syringaldehyde, Terpineol, Terpinolene Terpinyl acetate, Terpinyl butyrate, Terpinyl formate, Terpinyl isobutyrate, Terpinyl propionate, Tetrahydro-4-methyl-2-(2-methyl-1-propenyl)-2H-pyran, Tetrahydrofurfuryl acetate, Tetrahydrofurfuryl alcohol, Tetrahydrofurfuryl butyrate, Tetrahydrolinalool, Tetrahydromyrcenol, Thiazole, Thymol, Triacetin, Tributyl 2-acetylcitrate, Tricarballylic acid, Tridecanal, Tripropionin, Tris(methylthio)methane, Trithioacetone, Trivertal, Undecanal, Undecane, Undecanoic acid, Undecyl alcohol, Undecylenic acid, Valeraldehyde, Valeric acid, Vanillic acid, Vanillin acetate, Vanillin isobutyrate, Vanillin, Vanillyl butyl ether, Vanillylacetone, Whiskey lactone, Xylitol, α-Terpinene, α-Terpineol, cis-8-Undecenal, δ-Tetradecalactone, δ-Undecalactone, γ-Terpinene, γ-Undecalactone, o-Toluenethiol, p,α,α-Trimethylbenzyl alcohol, p-Tolualdehyde, p-Tolyl acetate, p-Tolyl phenylacetate, trans, trans-2,4-Undecadienal, trans-2-Tridecenal, and trans-2-Undecenal.

As used throughout this application, the term “pharmaceutically effective amount of a compound for pharmaceutical, veterinary, or agricultural use” shall mean an amount of that compound that exhibits the intended pharmaceutical (or veterinary, or agricultural) therapeutic effect when administered. Examples of methods of administration include, but are not limited to, oral administration (e.g., ingestion, buccal or sublingual administration), anal or rectal administration, topical applications, aerosol applications, inhalation, intraperitoneal administration, intravenous administration, transdermal administration, intradermal administration, subdermal administration, intramuscular administration, intrauterine administration, vaginal administration, administration into a body cavity, surgical administration (for example, at the location of a tumor or internal injury), administration into the lumen or parenchyma of an organ, and parenteral administration. The compositions can be administered in any form by any means. Examples of forms of administration include, but are not limited to, injections, solutions, creams, gels, implants, ointments, emulsions, suspensions, microspheres, powders, particles, microparticles, nanoparticles, liposomes, pastes, patches, capsules, suppositories, tablets, transdermal delivery devices, sprays, suppositories, aerosols, or other means familiar to one of ordinary skill in the art. In some embodiments, the compositions can be combined with other components. Examples include, but are not limited to, coatings, depots, matrices for time release and osmotic pump components.

As used throughout this application, the term “active” or “compound for pharmaceutical use” refers to any substance which, when administered to a human under conditions effective to cause absorption to the bloodstream, or cause a therapeutic or prophylactic effect.

A non-exhaustive list includes, but are not limited to, anesthetics, hypnotics, sedatives and sleep inducers, antipsychotics, antidepressants, antiallergics, antianginals, antiarthritics, antiasthmatics, antidiabetics, antidiarrheal drugs, anticonvulsants, antigout drugs, antihistamines, antipruritics, emetics, antiemetics, antispasmondics, appetite suppressants, neuroactive substances, neurotransmitter agonists, antagonists, receptor blockers and reuptake modulators, beta-adrenergic blockers, calcium channel blockers, disulfarim and disulfarim-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, and monoamine oxidase inhibitors. Therefore, as used throughout this application, the term “lipophilic compound for pharmaceutical use” refers to a lipophilic compound that is also a compound for pharmaceutical use. Examples of compounds or materials for pharmaceutical use include, but are not limited to, itraconazole, astemizole, saquinavir, amprenavir, paclitaxel, docetaxel, doxorubicin, ibuprofen, posaconazole, tacrolimus, danazol, estrogen, lopinavir, tamoxifen, nevirapine, efavirenz, delaviridine, nelfinavir, raloxifene, erythromycin, carbamazepine, ketoconazole, indinavir, progesterone, ritonavir, amiodarone, atorvastatin, azithromycin, carvedilol, chlorpromazine, cisapride, ciprofloxacin, cyclosporine, dapsone, diclofenac, diflunisal, flurbiprofen, glipizide, glyburide, griseofulvin, indomethacin, lansoprazole, mebendazole, naproxen, warfarin, terfenadine, talinolol, sirolimus, piroxicam, phentoin, and domperidone.

As used throughout this application, the term “active” or “compound for veterinary use” refers to any substance which, when administered to an animal under conditions effective to cause absorption to the bloodstream, or cause a therapeutic or prophylactic effect. A non-exhaustive list includes an Adjuvant, Anaesthetic, Analgesic, Antibiotic, Anticonvulsant agent, Antidote, Antiemetic, Antifungal, Anti-inflammatory, Antimicrobial, Antiprotozoal, Bactericide, Bee repellent, Behaviour modifier, Bloat remedy, Cardiovascular agent, CNS stimulant, Coccidiostat, Diagnostic Antigens, Diluent, Ectoparasiticide, Endocrine agent (hormone), Endoparasiticide, Euthanasia agent, Gastrointestinal tract modifier, Hormonal Growth Promotant, Immune Stimulant, Immunomodulator, Miticide, Musculoskeletal modifier, Obstetric Aid, Oral nutrient/electrolyte, Parenteral nutrient/electrolyte, Poultice, Renal & urinary tract modifier, Respiratory tract modifier, Sedative, Skin/coat Conditioner, and Vaccine. Therefore, as used throughout this application, the term “compound for veterinary use” refers to a compound that is also a compound for veterinary use. Examples of compounds or materials for veterinary use include, but are not limited to 1-(n-butylamino)-1-methylethyl-phosphonic acid, 2-hydroxy benzoic acid, 2-phenoxyethanol, 2-propenoic acid (polymer with 2-propenal), 4-androstene-3,17-dione, 9-alpha-fluoroprednisolone acetate, abamectin, acepromazine maleate, acetic acid, acetylglucosamine, acriflavine, adenosine 5 monophosphate, adenosine triphosphate, aglepristone, alanine, albendazole, alcohols (c10-16, ethoxylated; c12-15, ethoxylated; c12-c18, ethoxylated propoxylated), alfaxalone, Allantoin, allium sativum (ground garlic), aloe vera, alpha-cypermethrin, altrenogest, aluminium hydroxide, aluminium silicate, amitraz, ammonium chloride, amoxicillin, amoxycillin present as amoxycillin trihydrate, amoxycillin trihydrate, amphotericin b, ampicillin, ampicillin trihydrate, amprolium, aniseed oil, apomorphine hydrochloride, apramycin sulphate, atipamezol hydrochloride, atropine sulphate, avian encephalomyelitis virus (attenuated), avian gut flora, avian influenza type a, subtype h5n2, avian reovirus (attenuated), avian reovirus (inactivated), avilamycin, azaperone, bacitracin, Bacterial, yeast and fungal extracts, balsam peru, barium selenate, bee repellency aid, benazepril hydrochloride, bendiocarb, bentonite, benzalkonium chloride, benzenesulphonic acid, c10-16 alkyl derivs., Benzocaine, benzoic acid, benzoyl peroxide, benzylpenicillin sodium, beta-cyclodextrin, beta-estradiol, betamethasone, betamethasone valerate, biotin, bismuth salicylate, bismuth subnitrate, boracic acid, bordetella bronchiseptica (attenuated), bordetella bronchiseptica (inactivated), boric acid, bovine cartilage, bovine coronavirus (inactivated), bovine rotavirus (inactivated), bovine viral diarrhoea virus (inactivated), brewers yeast, bromhexine hydrochloride, bronopol, brotizolam, bupivacaine hydrochloride, buserelin acetate, butorphanol tartrate, caffeine, caffeine citrate, calamine, calcium, calcium borogluconate, calcium carbonate (limestone), calcium chloride, calcium chloride dehydrate, calcium copper edentate, calcium formate, calcium gluconate (d isomer), calcium gluconate (mixture of isomers), calcium glycerophosphate, calcium hydroxide, calcium hypophosphite, calcium iodate, calcium lactobionate, calcium oxide (lime), calcium pantothenate, calcium pentosan polysulphate, calcium phosphate dibasic, calcium phosphate tribasic, calcium propionate, camphor oil (light), campylobacter fetus subsp fetus (inactivated), campylobacter jejuni (inactivated), canine “appeasing” pheromones, canine (gamma) globulins, canine adenovirus type 2 (attenuated), canine distemper virus (attenuated), canine parainfluenza virus (attenuated), canine parvovirus (attenuated), canine parvovirus (inactivated), carbadox, carbaryl, carprofen, cartilage powder, casein, castor oil, cefadroxil monohydrate, cefovecin present as cefovecin sodium, cefquinome, ceftiofur, ceftiofur present as ceftiofur hydrochloride, ceftiofur present as ceftiofur sodium, cefuroxime sodium, centella asiatica concentrate, centella asiatica oil, cephalexin, cephalexin mono-hydrate, cephalonium, cephapirin benzathine, cetostearyl alcohol, cetrimide, cetrimonium bromide, chicken anaemia virus—live, chitosan, chlamydia psittaci, chloramine t, chlorfenvinphos, chlorhexidine, chlorhexidine Diacetate, chlorhexidine digluconate, chlorocresol, chloroxylenol, chlorpheniramine maleate, chlorpyrifos, chlortetracycline, chlortetracycline hydrochloride, cholecalciferol, choline, choline (45% in methanol), choline bitartrate, choline chloride, chondroitin, chondroitin sulphate, chromic chloride, citric acid (anhydrous), citric acid monohydrate, citrus pulp, clanobutin sodium, clavulanic acid, clavulanic acid present as potassium clavulanate, clenbuterol, clenbuterol hydrochloride, clindamycin hydrochloride, clomipramine hydrochloride, cloprostenol, cloprostenol present as cloprostenol sodium, cloprostenol sodium, clorsulon, closantel, closantel as the sodium salt, clostridium chauvoei toxoid/cells, clostridium haemolyticum toxoid/cells, clostridium novyi type b toxoid/cells, clostridium perfringens type a toxoid, clostridium perfringens type b toxoid/cells, clostridium perfringens type c toxoid, clostridium perfringens type d toxoid, clostridium septicum toxoid, clostridium sordellii toxoid, clostridium tetani toxoid, clotrimazole, cloxacillin benzathine, cloxacillin sodium (anhydrous), cloxacillin sodium monohydrate, cobalt, cobalt (ii) sulphate, cobalt carbonate, cobalt chloride hexahydrate, cobalt edta, cobalt gluconate, cobalt hydroxide, cobalt oxide (ii, iii), cobalt present as cobalt sulphate, cobalt present as disodium cobalt edta, cobalt sulphate heptahydrate, colostrums, contagious pustular dermatitis virus, copper, copper (i) oxide, copper (ii) oxide, copper disodium edta, copper edta, copper gluconate, copper hydroxide, copper indomethacin, copper naphthenate, copper present as calcium copper edentate, copper present as copper disodium edta, copper present as copper sulphate, copper present as cupric glycinate, copper sulfate pentahydrate, copper sulphate, coronavirus (inactivated), corynebacterium pseudotuberculosis exotoxin, coumaphos, creatinine monohydrate, csfv-e2 antigen, cupric chloride, cyclosporin a., cyfluthrin, cypermethrin, cyromazine, decoquinate, deer sinew, deer velvet, deet, delmadinone acetate, deltamethrin, dembrexine (hydrochloride), deracoxib, deslorelin, destiny prebiotic, detomidine hydrochloride, dexamethasone, dexamethasone-trimethyl acetate, dexamethasone 21-isonicotinate, dexamethasone present as dexamethasone acetate, dexpanthenol, dextrose, dextrose: glucose bp, d-glucitol, d-glucosamine, diazepam, diazinon, dibutyl phthalate, dichelobacter nodosus, dichlorobenzyl alcohol, dichlorophen, dicloxacillin sodium, dicyclanil, difloxacin hydrochloride, diflubenzuron, dihydrostreptomycin, dihydrostreptomycin present as dihydrostreptomycin sulphate, dihydrostreptomycin sulphate, diisopropylamine dichloroacetate, dimethyl phthalate, dimethyl sulphoxide, dimetridazole, dinoprost, di-n-propylisocinchomeronate, dipyrone, disodium citrate, disodium cobalt edta, disodium hydrogen phosphate, disodium manganese edta, disodium zinc edta, domperidone, doramectin, doxapram hydrochloride, doxycycline, doxycycline hyclate, doxycycline hydrochloride, doxycycline monohydrate, dye (methylene blue ci 52015), econazole nitrate (unspecified), edetic acid, egg drop syndrome virus, egg powder containg immunoglobulins, eimeria acervulina (attenuated) hp, eimeria brunetti (attenuated) hp, eimeria maxima (attenuated) cp, eimeria maxima (attenuated) mfp, eimeria mitis (attenuated) hp, eimeria necatrix (attenuated) hp, eimeria praecox (attenuated) hp, eimeria spp (live), eimeria tenella (attenuated) hp, eltenac, emodepside, emu oil, enrofloxacin, enzogenol, epidermal growth factor, epigallocatechin gallate, eprinomectin, equine arteritis virus, equine encephelomyelitis virus (inactivated)—eastern, equine encephelomyelitis virus (inactivated)—western, equine gamma globulins, equine influenza virus (inactivated), equine rhinopneumonitis virus (inactivated), equine somatotropin, erysipelothrix rhusiopathiae (inactivated), erythromycin, escherichia coli (inactivated), escherichia coli 987p (pili), escherichia coli k88ab (pili), escherichia coli k88ac (pili), escherichia coli k99 (pili), estradiol benzoate, etamiphylline camsylate, ethanol, ethohexadiol, ethoxy propoxy copolymer, ethyl lactate, ethylenediamine dihydroiodide, ethyloestrenol, etodolac, etofenprox, eucalyptus oil, febantel, feline calici virus (attenuated), feline calici virus (inactivated), feline chlamydia psittaci strain baker, feline herpes virus type 1, feline immunodeficiency virus petaluma strain, feline immunodeficiency virus shizuoka strain, feline leukaemia virus (inactivated), feline panleucopenia virus (attenuated), feline panleucopenia virus (inactivated), feline rhinotracheitis virus (attenuated), feline rhinotracheitis virus (inactivated), fenbendazole, fentanyl citrate, fenthion, ferric ammonium citrate, ferric chloride, ferric citrate, ferric glycerophosphate, ferric hydroxide, fipronil, firocoxib, florfenicolm, flubendazole, flumethrin, flunixin, flunixin meglumine, flunixin present as flunixin meglumine, flurogestone acetate, find inactivated virus, folic acid, follicle stimulating hormone-pituitary, formaldehyde, fowl laryngotracheitis virus (attenuated), fowl pox virus (attenuated), framycetin sulphate, frusemide, fucidin, furazolidone, fusidic acid hemi hydrate, gamma oryzanol, gentamicin, gentamycin sulphate, gentian violet, ginger, glucosamine hydrochloride, glucosamine sulphate, glucose, glycerol, glycine, glycopyrrolate, gnrf-protein conjugate, gonadorelin, gonadorelin present as gonadorelin acetate, gonadotrophin (gnrf poly albumin), green lip mussel, greers antigens, griseofulvin, guaiphenesin, gum resin, haemophilis influenza, haemophilus parasuis serovar 4 (inactivated), haemophilus parasuis serovar 4 (strain 2170b), haemophilus parasuis serovar 5 (inactivated), haemophilus parasuis serovar 5 strain ia 84-29755, haliotis iris, halofuginone base, halothane, harpagophytum procumbens, herbal oral nutritional compound, herbs n.o.s., hexetidine, histamine phosphate (anhydrous), hydrocortisone, hydrocortisone aceponate, hydroxocobalamin, hydroxocobalamin acetate, hydroxocobalamin hydrochloride, hydroxocobalamin present as hydroxocobalamin acetate, hydroxyprogesterone caproate, hydroxypropyl methylcellulose, hyoscine butylbromide, hyoscine hydrobromide, hyoscine hydrobromidetri-hydrate, hyosine methylbromide, imidacloprid, imidapril hcl, immunoglobulin, infectious bovine rhinotrocheitis virus (inactivated), infectious bronchitis virus (attenuated), infectious bronchitis virus (inactivated), infectious orf virus (nz7 strain), Ingred, Inositol, insulin, iodine, iodine present as an iodophor, iodine present as ethylenediamine dihydroiodide, iodophor, iron, iron dextran, iron present as iron dextran, isoeugenol, isoflurane, isoxsuprine hydrochloride, isoxsuprine lactate, ispaghula husk, ivermectin, japanese encephalitis virus, kaolin, ketamine as ketamine hydrochloride, ketamine hydrochloride, ketoprofen, klebsiella pneumonia, lactic acid, lactose, lanolin, lasalocid, lasalocid sodium, lawsonia intracellularis, l-carnitine, lecithin, leptospira borgpetersenii serovar hardjo, leptospira copenhageni, leptospira hardjo, leptospira interrogans serovar copenhageni (inactivated), leptospira interrogans serovar icterohaemorrhagiae, leptospira interrogans serovar Pomona, leptospira interrogans serovar tarassovi (hyos), leptospira Pomona, levamisole, levamisole hydrochloride, levamisole phosphate, L-glutamine, glycine, glucuronic acid, proline, mucopolysaccharides, glutamic acid, managanese sulphate, pyridoxine HCL (vitamin B6), ascorbic acid, copper sulphate, sulphur, lidocaine hydrochloride, lignocaine, lincomycin hydrochloride, lincomycin present as lincomycin sulphate, linseed oil, 1-methionine, lufenuron, luteinising hormone pituitary, lysine, maduramicin, magnesium, magnesium chloride, magnesium chloride hexahydrate, magnesium glycerophosphate, magnesium hydroxide, magnesium hypophosphite, magnesium oxide, magnesium phosphate tribasic, magnesium pidolate, magnesium sulfate, magnesium sulfate anhydrous, malachite green, maldison, malic acid, malto-dextrine, manganese (ii) sulfate monohydrate, manganese present as disodium manganese edta, manganese sulphate, marbofloxacin, marek's disease virus (attenuated), maropitant citrate, medetomidine hydrochloride, medroxyprogesterone acetate, megestrol acetate, melatonin, meloxicam, mepivacaine hydrochloride, mepyramine maleate, metacresolsulphonic acid/formaldehyde condensate, methandriol dipropionate, methyl-sulphonyl methane, methyl salicylate, methylprednisolone acetate, metronidazole, miconazole, miconazole nitrate, milbemycin oxime, mineral oil, petroleum distillates, solvent-refine, minimum essential medium (mem), molasses, monensin, monensin sodium, monoethanolamine, monosulfiram, montmorillonite, morantel, morantel citrate, morantel tartrate, moraxella bovis strain epp 63, moraxella bovis strain fla 64, moraxella bovis strain sah 38, moxidectin, mucopolysaccharide, Mucopolysaccharides, L-glutamine, glycine, DL-methionine, L-proline, L-alanine, L-arginine, glutamic, glucuronic ascorbic & aspartic acids, mn sulphate, L-serine, L-valine, L-histidine, L-threonine, L-tyrosine, vit. B6, L-isoleucine, cu sulphate, sulphur, mussel powder, mycobacterial cell wall fraction, mycobacterium avium, mycobacterium bovis, mycobacterium paratuberculosis, mycoplasma gallisepticum, mycoplasma hyopneumoniae strain p-5722-3, mycoplasma synoviae, n-(2-ethylhexyl)-bicyclo[2,2,1]-5-heptene-2,2-dicarboximide, nafcillin sodium, naloxone hydrochloride, nandrolone decanoate, nandrolone laurate, narasin, neomycin, neomycin present as neomycin sulphate, neomycin sulphate, neomycin undecylenate, neospora caninum, niacinamide, nicarbazin, niclosamide, nicotinic acid, nimesulide, nitenpyram, nitrofurazone, nonyl phenol ethoxylate iodine, nonyl phenyl ethoxylate, norethandrolone, novobiocin sodium, nux vomica, nystatin, octyl dimethyl p-aminobenzoate, oestradiol, oestriol, oils and waxes, oleandomycin, omeprazole, oral nutritional compound, orange oil, orbifloxacin, orthophenyl phenol, oxantel pamoate, oxfendazole, oxibendazole, oxytetracycline, oxytetracycline dehydrate, oxytetracycline hydrochloride, oxytetracycline present as oxytetracycline dehydrate, oxytetracycline present as oxytetracycline hydrochloride, oxytocin, panthenol, paradichlorobenzene, paraffin wax, parainfluenza-3 virus (inactivated), pasteurella haemolytica (inactivated), pasteurella multocida (inactivated), peanut oil, pectin, peg-7 glyceryl cocoate, penethamate hydriodide, penicillin benethamine, penicillin g benzathine, penicillin g potassium, penicillin g procaine, penicillin procaine, pentobarbitone sodium, peppermint oil, permethrin, pethidine hydrochloride, petrolatum, phenobarbital sodium, phenobarbitone, phenol, phenylbutazone, phenylpropanolamine hydrochloride, phosphoric acid, phosphoryl ethanolamine, phosphorylethanolamine, pimobendan, piperonyl butoxide, pirlimycin hydrochloride, poly(oxy-1,2-ethanediyl), alpha-(4-nonylphenyl)-omega-hydroxy-, branched, polyandroalbumin, polymyxin, polymyxin b sulfate, polyoxalene, polyoxyethylene alcohol, polyoxyethylene sorbitan mono-oleate, polypropylene oxide; polyethylene oxide, polysulphated glycosaminoglycan, porcine circovirus type 2 orf-2 protein, porcine circovirus type1-type2 chimera (inactivated), porcine parvovirus (inactivated), porcine somatotrophin, porphyromonas denticanis (inactivated), porphyromonas gulae (inactivated), porphyromonas salivosa, potassium aluminium sulphate, potassium bicarbonate, potassium bromide, potassium chloride, potassium citrate, potassium citrate monohydrate, potassium clavulanate, potassium glycerophosphate, potassium iodate, potassium iodide, potassium phosphate monobasic, potassium sulphate, povidone iodine, praziquantel, prednisolone, prednisolone acetate, Probiotics, progesterone, Proligestone, propantheline bromide, Propentofylline, propetamphos, propionibacterium acnes, propofol, propoxur, propylene glycol, prostianol, proteus vulgaris, pseudomonas aeruginosa, psyllium hydrophilic mucilloid, pyrantel pamoate, pyrethrin ii, pyrethrins, pyridoxine hydrochloride, pyriprole, pyriproxyfen, quinine sulphate, rabbit calicivirus (rcd)—inactivated, rabies virus (inactivated), racementhol, racemethionine, ractopamine hydrochloride, recombinant feline omega interferon, reserpine, retinol acetate, riboflavin 5′-phosphate sodium, rice, ricobendazole, robenidine hydrochloride, romifidine, ronidazole, rotavirus (inactivated), rotenone, roxarsone, s-adenosylmethionine, salinomycin, salinomycin sodium, salix alba extract, salmonella bovis morbificans cnl412 (inactivated), salmonella brandenburg lbr 3684 (inactivated), salmonella hindmarsh cn 5989 (inactivated), salmonella sp, salmonella typhimurium, salmonella typhimurium cn5988 (inactivated), salmonella typhimurium cn6141 (inactivated), sassafras oil, selamectin, selenium, selenium dioxide, selenium edta, selenium present as barium selenate, selenium present as selenium edta, selenium present as sodium selenate, selenium sulphide, semduramicin sodium, shark cartilage, shigella dysenteriae, silicon oestradiol suspension (20%), silver sulfadiazine, slippery elm powder, s-methoprene, sodium acetate, sodium acid pyrophosphate, sodium alpha-hydroxybenzylphosphinate, sodium ampicillin, sodium bicarbonate, sodium cacodylate, sodium carbonate, sodium chloride, sodium chondroitin sulphate, sodium citrate, sodium diacetate, sodium dichloroisocyanurate, sodium dihydrogen phosphate, sodium edentate, sodium glycerophosphate, sodium hyaluronate, sodium hydroxide, sodium lactate, sodium pentosan polysulphate, sodium phenytoin, sodium propionate, sodium salicylate, sodium selenate, sodium selenite, sodium sulphate, sodium tripolyphosphate, sorbitol, spectinomycin dihydrochloride pentahydrate, spectinomycin sulphate, spinosad, spiramycin, stanozolol, staphylococcus sp, starch, streptococcus equi, streptococcus equi (live), streptococcus sp, streptococcus suis (inactivated), streptomycin, streptomycin sulphate, streptomycin, dihydro, sulphate, strychnine, sulfadiazine, sulphadimethoxine sodium, sulphaguanidine, sulphamerazine, sulphamethazine, sulphamethoxypyridazine, sulphanilamide, sulphapyridine, sulphaquinoxaline, sulphathiazole, sulphisoxazole, sulphur, synthetic feline facial hormone, tannic acid, tea tree oil, temephos, tepoxalin, tergitol np-9 surfactant, teric bl8, testosterone propionate, tetanus antitoxin, tetanus toxoid, tetrachlorvinphos, tetracycline hydrochloride, tetramisole hydrochloride, thiamine disulphide, thiamine hydrochloride, thioctic acid, thiopentone sodium, thiostrepton, tiamulin hydrogen fumarate, tiletamine hydrochloride, tilmicosin, tilmicosin as tilmicosin phosphate, titanium dioxide, tolazoline, tolfenamic acid, toltrazuril, toxoplasma gondii, tragacanth, tranexamic acid, trenbolone acetate, triamcinolone acetonide, trichlorfon, trichlormethiazide, triclabendazole, triflumuron, trimethoprim, trisodium citrate, trisodium citrate dehydrate, trypsin, turkey herpes virus (attenuated), turpentine, tylosin, tylosin factors b+c+d, undecylenic acid, virginiamycin, vitamin a, vitamin a palmitate, vitamin b1 (thiamine), vitamin b12, vitamin b2 (riboflavin), vitamin b6 (pyridoxine hydrochloride), vitamin c (ascorbic acid), vitamin e, vitamin e acetate, vitamin k1, vitamin k3 (menadione sodium bisulphite), water, xylazine, xylazine hydrochloride, xylazine present as xylazine hydrochloride, xylitol, yeast (inactivated), yersinia pseudotuberculosis, yohimbine hydrochloride, yucca shidigera plant extract, zinc, zinc bacitracin, zinc edentate, zinc oxide, zinc sulphate, zinc sulphate heptahydrate, zinc sulphate monohydrate, zolazepam hydrochloride

As used throughout this application, the term “active” or “compound for agricultural use” refers to any substance administered to a plant. A non-exhaustive list includes an Anti-sapstain, Bactericide, Fungicide, Herbicide, Insecticide, Miticide, Molluscicide, Nematicide, Nematicide, Pheromones, Plant growth regulator, and Vertebrate Toxic Agent. Examples of compounds or materials for agricultural use include, but not limited to 1,3-dichloropropene, 1,4-dimethylnaphthalene, 1-methyl cyclopropene, 1-naphthylacetic acid, 2,2-dichloropropionic acid, 2-hydroxy benzoic acid, 3-bromo-1-chloro-5,5-dimethylhydantoin, 3-chloro-p-toluidine hydrochloride, 8-hydroxyquinoline sulphate, Abamectin, acephate, acetochlor, agrobacterium radiobacter, alachlor, alpha-cypermethrin, aluminium phosphide, aminopyralid, amitraz, amitrole, ammonium thiosulphate, asulam, atrazine, aviglycine hydrochloride, azaconazole, azadirachtin, azinphos-methyl, azoxystrobin, bacillus subtilis, bacillus subtilis qst 713, bacillus thuringiensis var aizawai (abbott 1857), bacillus thuringiensis var aizawai/kurstaki, bacillus thuringiensis var kurstaki (h-3a,3b hdl), bacillus thuringiensis var kurstaki (h-3a,3b, hd 263), bacillus thuringiensis var kurstaki (h-3a,3b, sa-1 1), beauvaria bassiana (k4b1), benalaxyl, benomyl, bentazone, benzalkonium chloride, bifenthrin, bordeaux mixture, boscalid, brodifacoum, bromacil, bromadiolone, bromopropylate, bromoxynil, bupirimate, buprofezin, calcium polysulfide, canola oil, captan, carbaryl, carbendazim, carbon dioxide, carboxin, carfentrazone-ethyl, chitosan, chloralose, chlorethephon, chloridazon, chlorimuron-ethyl, chlormequat-chloride, chloropicrin, chlorothalonil, chlorpropham, chlorpyrifos, chlorsulfuron, chlorthal-dimethyl, cholecalciferol, clethodim, clodinafop-propargyl, clofentezine, clomazone, clopyralid, clopyralid present as clopyralid monoethanolamine, clothianidin, copper, copper (i) oxide, copper ammonium acetate, copper hydroxide, copper oxychloride, copper sulphate, corn cob, powdered, coumatetralyl, cresol (all isomers), cyanazine, cydia pomonellla granulosis virus, mexican strain, cyfluthrin, cymoxanil, cypermethrin, cyproconazole, cyprodinil, cyromazine, daminozide, dazomet, deltamethrin, desmedipham, diazinon, dicamba, dichlobenil, dichlorprop-p, dichlorvos, dicloran, dicofol, difenoconazole, diflubenzuron, diflufenican, dimethenamid, dimethoate, dimethomorph, diphacinone, diquat, diquat present as diquat dibromide, dithianon, diuron, dodine, emamectin benzoate, endosulfan, endothal, epoxiconazole, esfenvalerate, ethofumesate, ethyl formate, etridiazole, fatty acids, fatty acids (potassium salts), fenamidone, fenamiphos, fenarimol, fenhexamid, fenitrothion, fenoxaprop-p-ethyl, fenpropidin, fenpropimorph, fenpyroximate, fipronil, flazasulfuron, flocoumafen, fluazifop-p-butyl, fluazinam, fludioxonil, flumethrin, flumetsulam, fluoxastrobin, fluroxypyr, flusilazole, flusulfamide, flutriafol, folpet, forchlorfenuron, fosetyl-aluminium, fuberidazole, furathiocarb, gibberellic acid, gibberellin a4/a7, glufosinate-ammonium, glyphosate, glyphosate present as glyphosate potassium and glyphosate triethanolamine, glyphosate present as glyphosate-potassium, halosulfuron-methyl, haloxyfop, haloxyfop [(r)-isomer], hexazinone, hydrogen cyanamide, hydrogen cyanide, imazalil, imazapyr, imazethapyr, imidacloprid, indolebutyric acid, indoxacarb, iodocarb, iodosulfuron-methyl-sodium, ioxynil, iprodione, iprovalicarb, iron phosphate, iron sodium edta, isoproturon, kresoxim-methyl, lambda-cyhalothrin, lecanicillium lecanii (strain k4vl), lecanicillium lecanii blastospores (strain k4v2), l-flamprop-isopropyl, linuron, lubricating oils, petroleum, c15-30, hydrotreated neutral oil-based, contg. solvent deasphalted residual oil, lufenuron, magnesium phosphide, maldison, maleic hydrazide, mancozeb, mandipropamid, mcpa, mcpb, mecoprop, mecoprop-p, mepiquat-chloride, mesotrione, metalaxyl, metalaxyl-m, metaldehyde, metam sodium, metamitron, methabenzthiazuron, methamidophos, methiocarb, methomyl, methoxyfenozide, methyl bromide, methyl canolate, metiram, metribuzin, metsulfuron-methyl, milbemectin, mineral oil, myclobutanil, n6-benzyladenine, neem seed kernel extract, nicosulfuron, novaluron, oils-mineral-insecticidal, oleic acid, oryzalin, oxadiazon, oxamyl, oxyfluorfen, paclobutrazol, palm oil derived fatty acids, pantoea agglomerans, strain p10c, paraffin oil, paraquat, paraquat present as paraquat dichloride, penconazole, pencycuron, pendimethalin, permethrin, phenmedipham, phorate, phosphorous acid, phosphorus, picloram, picloram present as picloram monoethanolamine, picloram present as picloram triethanolamine, picoxystrobin, pindone, pine oil, pinoxaden, piperonyl butoxide, pirimicarb, pirimiphos-methyl, potassium bicarbonate, potassium cyanide, primisulfuron-methyl, prochloraz, procymidone, prohexadione-calcium, prometryn, propachlor, propamocarb, propargite, propazine, propham, propiconazole, propineb, propyzamide, prothioconazole, prothiofos, pymetrozine, pyraclostrobin, pyrethrins, pyridate, pyrimethanil, quinoxyfen, quintozene, quizalofop-p-ethyl, rabbit calicivirus (rcd), rotenone, serratia entomophila (strain 626), sethoxydim, simazine, s-metolachlor, sodium cyanide, sodium fluoroacetate, sodium tetrathiocarbonate, spinetoram, spinosad, spiromesifen, spirotetramat, spiroxamine, steinernema feltiae, sulfentrazone, sulphur, sulphur present as poysulphide sulphur, tau-fluvalinate, tca, tebuconazole, tebufenozide, terbacil, terbufos, terbuthylazine, terbutryn, thiabendazole, thiacloprid, thiamethoxam, thidiazuron, thifensulfuron-methyl, thiodicarb, thiophanate-methyl, thiram, thymol, tolclofos-methyl, tolylfluanid, tralkoxydim, triadimefon, triadimenol, tri-allate, tribenuron-methyl, trichlorfon, trichoderma atroviride (lc52), trichoderma harmatum, trichoderma harzianum rifai (5 strains), triclopyr, triclopyr butoxyethyl ester: triclopyr bee, trifloxystrobin, trifluralin, triforine, trinexapac-ethyl, ulocladium oudemansii, and ziram.

The term “solvate” refers to the compound formed by the interaction of a solvent and a compound. Suitable solvates are pharmaceutically or agriculturally acceptable solvates, such as hydrates, including monohydrates and hemi-hydrates. “Pharmaceutically or agriculturally acceptable salt” refers to a salt of a compound that is pharmaceutically acceptable and that possesses the desired pharmacological activity of the parent compound. Such salts may include: (i) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (ii) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

Increased bioavailability can include any mechanism that has a desired effect on cellular efflux, cellular influx, or clearance. “Clearance” includes any type of elimination of one or more compounds from cells, blood, plasma, tissues or organs (e.g. intestinal clearance, hepatic clearance, renal clearance, and pulmonary clearance each describe elimination of compounds from the blood). Clearance may be described via the observed differences of renal excretion and elimination by all other processes including influx and efflux mechanisms (e.g. gastrointestinal clearance, excretory clearance, biliary clearance and enterohepatic cycling, metabolic clearance). Examples of systemic fluids include, but are not limited to: blood; cerebrospinal fluid; lymph; and any other tissue fluids (including increased amounts in tissues that are bathed by such fluids, such as the brain, tissue of one or more visceral organs, connective tissue, muscle, fat, or one or more tissues in the skin). In some embodiments, the increase is systemic, as in the case of an increase measurable anywhere in the blood. In some embodiments, the increase is more localized, as is the case with some embodiments involving topical administration in which the increase is measured only in areas near the administration. An increase in portion of the dosage that reaches a fluid or tissue measurable by any reliable means is within this definition, including but not limited to increases identified by measuring the total systemic drug concentration over time after administration. In some embodiments, concentrations are determined by measuring the tissue or fluids themselves, or by measuring fractions thereof (for example, without limitation, serum or plasma in the case of blood). In some embodiments, increases for compounds that are excreted metabolized and/or un-metabolized in urine are determined by measuring levels of compounds or metabolites of the compounds in urine and will reflect an increase in systemic concentrations. In some embodiments an increase in compound bioavailability is defined as an increase in the Area Under the Curve (AUC). AUC is an integrated measure of systemic compound concentrations over time in units of mass-time/volume and is measured from the time compound is administered (time zero) to infinity (when no compound(s) remaining in the body can be measured). Information regarding monitoring substances within a subject are known to persons of ordinary skill in the art and may be found in references such as M. Rowland and T. N. Tozer, Clinical Pharmacokinetics Concepts and Applications (third Ed., 1995), Lippincott Willams and Wilkins, Philadelphia. In some embodiments, the compounds or compositions of the invention may increase the bioavailability of a lipophilic compound by a factor of 0.1 to 10 in comparison to the lipophilic compound alone. In certain embodiments, the increase may be by a factor of 0.1 to 10, 1 to 9, 2 to 8, 3 to 7, 4 to 6, or about 5.

In some embodiments, the sulfo-polyester polymer powder(s), sulfo-polyester polymer powder blends, or compositions of the present invention are administered to persons or animals or plants to provide substances in any dose range that will produce desired physiological or pharmacological or agricultural results. Dosage will depend upon the substance or substances administered, the therapeutic endpoint desired, the desired effective concentration at the site of action or in a body fluid, and the type of administration. Information regarding appropriate doses of substances are known to persons of ordinary skill in the art and may be found in references such as L. S. Goodman and A. Gilman, eds, The Pharmacological Basis of Therapeutics, Macmillan Publishing, New York, and Katzung, Basic & Clinical Pharmacology, Appleton & Lang, Norwalk, Conn. (6.sup.th Ed. 1995). In some embodiments, the compounds and compositions of the present invention may be administered to a subject. Suitable subjects include a cell, population of cells, tissue or organism. In certain embodiments, the subject is a mammal such as a human. The compounds may be administered in vitro or in vivo.

The sulfo-polyester polymer powder(s), sulfo-polyesters polymer powder blends, and compositions of the present invention may also be used to increase the bioavailability of a compound(s) when co-administered. In some embodiments, the sulfo-polyester polymer powder(s), sulfo-polyester polymer powder blends, compositions of the present invention and the compound may be administered at the same time. This may be accomplished, for example, by administering them together as separate compounds or as one composition. In other embodiments, the sulfo-polyester polymer powder(s), sulfo-polyester polymer powder blends, and compositions of the present invention may be administered before the compound(s).

One of skill in the art may determine an increase in the bioavailability of a compound using assays standard in the art. For example, the plasma or tissue concentration of a lipophilic compound in an animal may be determined after administration of the lipophilic compound alone or after administration of the lipophilic compound in combination with a composition of the present invention.

The invention includes methods in which one or more of the sulfo-polyester polymer powders and blends thereof of the present invention is co-administered with one or more lipophilic compounds in the presence and/or absence of other commonly used excipients. In some embodiments, the lipophilic compound(s) is (are) a lipophilic compound for pharmaceutical use. In some embodiments, pharmaceutically effective amounts of the lipophilic compound(s) for pharmaceutical or agricultural use is (are) coadministered with one or more sulfo-polyester polymer and may be in the presence and/or absence of other commonly used excipients.

The invention includes methods in which one or more of the sulfo-polyester polymer powders, and/or blends thereof are an admixture or otherwise combined with one or more lipophilic compounds and may be in the presence or absence of commonly used excipients; for example, but not limited to: i) diluents such as lactose, dextrose, sucrose, sorbitol, mannitol, cellulose, and the like; ii) binders such as starch paste, gelatin, magnesium aluminum silicate, methylcellulose, sodium carboxymethyl-cellulose, polyvinylpyrrolidone and the like; iii) lubricants such as stearic acid, talcum, silica, polyethylene glycol, polypropylene glycol and the like; iv) absorbents, colorants, sweeteners and the like; v) disintegrants, such as effervescent mixtures and the like. Furthermore, said invention includes compositions prepared using conventional mixing, granulating, or coating methods and may contain 0.1% to 90% of the active ingredients. In some embodiments, the one or more lipophilic compounds are for pharmaceutical use. Such methods can be used, for example, to prepare a bioenhanced pharmaceutical composition in which the solubility of the lipophilic compound(s) is (are) enhanced. In some embodiments, the resulting compositions contain a pharmaceutically effective amount of a lipophilic compound for pharmaceutical use. The resulting compositions (formulations) may be presented in unit dosage form and may be prepared by methods known in the art of pharmacy. All methodology includes the act of bringing the active ingredient(s) into association with the carrier which constitutes one or more ingredients. Therefore, compositions (formulations) are prepared by blending active ingredient(s) with a liquid carrier or a finely divided solid carrier, and/or both, and then, if needed, shaping the product into a desired formulation.

In some embodiments, the compositions of the present invention contain one or more additional desirable components or compounds. Any desirable compounds can be used. Examples include, but are not limited to, additional active pharmaceutical ingredients as well as excipients (e.g. cyclodextrins), diluents, and carriers such as fillers and extenders (e.g., starch, sugars, mannitol, and silicic derivatives); binding agents (e.g., carboxymethyl cellulose and other cellulose derivatives, alginates, gelatin, and polyvinyl-pyrrolidone); moisturizing agents (e.g., glycerol); disintegrating agents (e.g., calcium carbonate and sodium bicarbonate); agents for retarding dissolution (e.g., paraffin); resorption accelerators (e.g., quaternary ammonium compounds); surface active agents (e.g., cetyl alcohol, glycerol monostearate); adsorptive carriers (e.g., kaolin and bentonite); emulsifiers; preservatives; sweeteners; stabilizers; antioxidants; buffers; bacteriostats; coloring agents; perfuming agents; flavoring agents; lubricants (e.g., talc, calcium and magnesium stearate); solid polyethyl glycols; and mixtures thereof. Examples of carriers include, without limitation, any liquids, liquid crystals, solids or semi-solids, such as water or saline, gels, creams, salves, solvents, diluents, fluid ointment bases, ointments, pastes, implants, liposomes, micelles, giant micelles, and the like, which are suitable for use in the compositions.

Topical application to skin sites is accomplished in association with a carrier, and particularly one in which the active ingredient is soluble per se or is effectively solubilized (e.g., as an emulsion or microemulsion). Where employed, the carrier is inert in the sense of not bringing about a deactivation or oxidation of active or adjunct ingredient(s), and in the sense of not bringing about any adverse effect on the skin areas to which it is applied. For example, the compounds according to the present invention are applied in admixture with a dermatologically acceptable carrier or vehicle (e.g., as a lotion, cream, ointment, soap, stick, or the like) so as to facilitate topical application and, in some cases, provide additional beneficial effects as might be brought about, e.g., by moisturizing of the affected skin areas. While the carrier for dermatological compositions can consist of a relatively simple solvent or dispersant such as water, it is generally preferred that the carrier comprise a composition more conducive to topical application. In particular, a dermatological composition which will form a film or layer on the skin to which it is applied so as to localize the application and provide some resistance to washing off by immersion in water or by perspiration and/or aid in the percutaneous delivery of the active agent. Many preparations are known in the art, and include lotions containing oils and/or alcohols and emollients such as olive oil, hydrocarbon oils and waxes, silicone oils, other vegetable, animal or marine fats or oils, glyceride derivatives, fatty acids or fatty acid esters or alcohols or alcohol ethers, lecithin, lanolin and derivatives, polyhydric alcohols or esters, wax esters, sterols, phospholipids and the like, and generally also emulsifiers (nonionic, cationic, or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophilic colloids. Such compositions are referred to herein as dermally, dermatologically, or pharmaceutically acceptable carriers.

“Therapeutically effective amount” or “effective amount” refers to the amount of a compound that, when administered to a subject for treating a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, or inhibiting at least one physical parameter which may not be discernible to the subject. Further, “treating” or “treatment” refers to delaying or preventing the onset or reoccurence of the disease or disorder or at least symptoms thereof in a subject which may be exposed to or predisposed to or may have previously suffered from a disease or disorder even though that subject does not yet experience or display symptoms of the disease or disorder.

Typical compositions of the invention contain up to about 90% by weight carrier, surfactant, and/or active. For example, the compositions can contain from about 90% to about 0.1% by weight, from about 40% to about 0.5% by weight, from about 20% to about 1% by weight, or from about 10% to about 2% by weight by weight, carrier, surfactant and/or active. Lower concentrations may be employed for less pronounced conditions (e.g. hyperpigmentation and in sunscreens and sunblocks used after skin brightening treatment) and higher concentrations may be employed with more acute conditions. The effective amount of compounds or compositions of the invention may range from about 0.1 to 200 milligrams (mg) per kilogram (kg) of subject weight. In certain embodiments, the compounds or compositions of the invention are administered at from about 200 mg/kg to 0.1 mg/kg or from about 100 mg/kg to 1.0 mg/kg, from about 50 mg/kg to 2 mg/kg, or from about 25 mg/kg to 5 mg/kg.

It should be understood that the ingredients particularly mentioned above are merely examples and that some embodiments of formulations comprising the compositions of the present invention include other suitable components and agents. The compositions of the invention may be used for, among other things, pharmaceutical and cosmetic purposes and may be formulated with different ingredients according to the desired use.

The invention further includes packages, vessels, or any other type of container that contain either an sulfo-polyester polymer powder(s), blends thereof, dispersions thereof of the present invention, or any composition comprising a sulfo-polyester polymer powder formulation of the present invention.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternative powder preparation methods (e.g. spray-drying, jet milling, spray freeze-drying, fluidized-bed spray coating, or supercritical fluid methods), particle size modifications (e.g. milling), and equivalents as may be included within the spirit and scope of the embodiments of the present disclosure as defined by the appended claims.

### EXAMPLES

This invention can be further illustrated by the following examples, although it will be understood that these examples are included merely for purposes of illustration and are not intended to limit the scope of the invention unless otherwise specifically indicated.

### Materials

AQ29D, AQ38S, AQ48S, AQ55S, Eastek 1200 (aka AQ65D), AQ1045S, AQ1350S, AQ1950S, EASTONE, and TPGS-1000 were obtained from Eastman Chemical Company. The “S” or “D” nomenclature used below refers to the solid or dispersed form of the polymer, respectively. Therefore, AQ29 and Eastek 1200 were obtained as dispersions; Eastek 1200 dispersion contains 2% propanol. HPLC grade water, ethanol, DMSO, kojic acid, vitamin E, hydroquinone, glycolic acid, and salicylic acid were purchased from Sigma-Aldrich (St. Louis, Mo., USA). A VWR SympHony SB20 pH meter was used (Oak Ridge, Tenn.). A Virtis (SP Industries Inc.; Warminster, Pa.) laboratory freeze dryer (model #4KBTXL-75) was used.

### Dispersion Preparations:

Eastman AQ Polymer dispersions have been used in Cosmetics and Personal Care applications (e.g. color cosmetics, sunscreen sprays and lotions, and hair styling products) and used in applications where removal of the adhesive after application is required (e.g. re-pulping of paper and plastic and glass recycling). Therefore, depending upon the desired dispersion and/or concentration to be prepared, different solvents (i.e. water, alcohol, etc) and mixing temperatures may be used.

### AQ38 Dispersion

AQ38 pellets (120 g) were weighed out into a beaker (100 mL). Water (280 mL) and a stir-bar were added to a beaker (500 mL). The water was heated. With continued heating, AQ38 was added (1.5 h) in portions with vigorous stirring. After an additional 30 min, the polymer appeared to be completely dispersed and was poured into a glass jar, allowed to slowly cool (6 h) to afford a milky white dispersion and capped. The next day, after mixing to ensure a homogenous dispersion, the pH at room temperature was taken (6.0±0.1). These methods afford an estimated 30% 10 AQ38 dispersion.

### AQ48 Dispersion

AQ48 pellets (128 g) were weighed out into a beaker (250 mL). Water (428 mL) and a stir-bar were added to a beaker (1000 mL). The water was heated. With continued heating, AQ48 was added (1.5 h) in portions with vigorous stirring. After an additional 30 min, the polymer appeared to be completely dispersed and was poured into a glass jar, allowed to slowly cool (6 h) to afford a thick hazy light yellow, but translucent, dispersion and capped. The next day, after mixing to ensure a homogenous dispersion, the pH at room temperature was taken (5.6±0.1). These methods afford an estimated 23% AQ48 dispersion.

### AQ55 Dispersion

AQ55 pellets (120 g) were weighed out into a beaker (100 mL). Water (280 mL) and a stir-bar were added to a beaker (500 mL). The water was heated. With continued heating, AQ55 was added (1.5 h) in portions with vigorous stirring. After an additional 30 min, the polymer appeared to be completely dispersed and was poured into a glass jar, allowed to slowly cool (6 h) to afford a thick translucent dispersion and capped. The next day, after mixing to ensure a homogenous dispersion, the pH at room temperature was taken (6.2±0.1). These methods afford an estimated 30% AQ55 dispersion.

### AQ1045 Dispersion

AQ 1045 block (yellow-orange sticky solid) was carefully cooled with liquid N₂ and pieces were carefully chipped off using a hammer and chisel. Afterwards, AQ1045 (120 g) was weighed out into a beaker (100 mL). Water (280 mL) and a stir-bar were added to a beaker (500 mL). The water was heated. With continued heating, AQ1045 was added (1.5 h) in portions with vigorous stirring. After an additional 30 min, the polymer appeared to be completely dispersed and was poured into a glass jar, allowed to slowly cool (6 h) to afford a milky white dispersion and capped. The next day, after mixing to ensure a homogenous dispersion, the pH at room temperature was taken (5.6±0.1). These methods afford an estimated 30% AQ 1045 dispersion.

### AQ1350 Dispersion

AQ1350 block (yellow-orange sticky solid) was carefully cooled with liquid N₂ and pieces were carefully chipped off using a hammer and chisel. Afterwards, AQ1350 (85 g) were weighed out into a beaker (100 mL). Water (320 mL) and a stir-bar were added to a beaker (500 mL). The water was heated. With continued heating, AQ1350 was added (1.5 h) in portions with vigorous stirring. After an additional 30 min, the polymer appeared to be completely dispersed and was poured into a glass jar, allowed to slowly cool (6 h) to afford a milky white dispersion and capped. The next day, after mixing to ensure a homogenous dispersion, the pH at room temperature was taken (5.4±0.1). These methods afford an estimated 21% AQ1350 dispersion.

### AQ1950 Dispersion

AQ1950 block (yellow-orange sticky solid) was carefully cooled with liquid N₂ and pieces were carefully chipped off using a hammer and chisel. Afterwards, AQ1950 (85 g) was weighed out into a beaker (100 mL). Water (320 mL) and a stir-bar were added to a beaker (500 mL). The water was heated to nearly boiling. With continued heating, AQ1950 was added (1.5 h) in portions with vigorous stirring. After an additional 30 min, the polymer appeared to be completely dispersed and was poured into a glass jar, allowed to slowly cool (6 h) to afford a milky white dispersion and capped. The next day, after mixing to ensure a homogenous dispersion, the pH at room temperature was taken (5.7±0.1). These methods afford an estimated 21% AQ1950 dispersion.

### AQ2150 Dispersion

AQ2150 block (yellow-orange sticky solid) was carefully cooled with liquid N₂ and pieces were carefully chipped off using a hammer and chisel. Afterwards, AQ2150 (60 g) was weighed out into a beaker (100 mL). Water (336 mL) and a stir-bar were added to a beaker (500 mL). The water was heated. With continued heating, AQ2150 was added (1.5 h) in portions with vigorous stirring. After an additional 30 min, the polymer appeared to be completely dispersed and was poured into a glass jar, allowed to slowly cool (6 h) to afford a hazy light yellow, but translucent, dispersion and capped. The next day, after mixing to ensure a homogenous dispersion, the pH at room temperature was taken (5.5±0.1). These methods afford an estimated 15% AQ2150 dispersion.

### AQ2350 Dispersion

AQ2350 block (yellow-orange sticky solid) was carefully cooled with liquid N₂ and pieces were carefully chipped off using a hammer and chisel. Afterwards, AQ2350 (50 g) was weighed out into a beaker (100 mL). Water (243 mL) and a stir-bar were added to a beaker (500 mL). The water was heated. With continued heating, AQ2350 was added (1.5 h) in portions with vigorous stirring. After an additional 30 min, the polymer appeared to be completely dispersed and was poured into a glass jar, allowed to slowly cool (6 h) to afford a hazy light yellow, but translucent, dispersion and capped. The next day, after mixing to ensure a homogenous dispersion, the pH at room temperature was taken (5.5±0.1). These methods afford an estimated 19% AQ2350 dispersion.

### EASTONE Dispersion

EASTONE pellets (121 g) were weighed out into a beaker (100 mL). Water (350 mL) and a stir-bar were added to a beaker (500 mL). The water was heated. With continued heating, EASTONE was added (1.5 h) in portions with vigorous stirring. After an additional 30 min, the polymer appeared to be completely dispersed and was poured into a glass jar, allowed to slowly cool (6 h) to afford a milky white dispersion and capped. The next day, after mixing to ensure a homogenous dispersion, the pH at room temperature was taken (6.2±0. 1). These methods afford a estimated 25-26% EASTONE dispersion.

### AQ Powder Preparation: Dispersion Freeze Drying (Lyophilize):

Individually, the AQ29, AQ38, AQ48, AQ55, Eastek 1200 (AQ65), AQ1045, AQ1350, AQ1950, AQ2150, AQ2350, and EASTONE dispersions were mixed and samples poured into 250 mL round bottom flasks. In order to get the amount of added dispersion, the flasks were weighed before and after addition. The dispersions were then swirled and cooled with a dry-ice bath until frozen. Afterwards, the materials were lyophilized overnight to afford white to off-white powders (see Table 1); after removal, some of the powders became clear or were a sticky mass and presumed to be a function of retained water not removed, i.e. greater mass than the estimated polymer percentage.

The materials were then transferred out of the round bottoms into individual glass jars and capped. The results for the solids obtained after the samples were lyophilized afforded a way to measure the actual polymer dispersion percentage. Those results were as follows: AQ29 (29.7%), AQ38 (29.1%), AQ48 (22.5%), AQ55 (28.2%), Eastek 1200 (AQ65; 35.7%), AQ1045 (30.7%), AQ1350 (22.7%), AQ1950 (24.6%), AQ2150 (16.0%), AQ2350 (21.0%), and EASTONE (24.9%). Having these numbers in hand, dispersion blends were subsequently prepared by appropriate dispersion ratio mixing, freezing, and lyophilized. Examples of the various polymer blends are summarized in Table 2. Furthermore, it was not until one approached around 25% of a sticky AQ polymer (i.e. AQ1045, AQ1350, AQ1950, AQ2150, AQ2350) before a powder blend became difficult to handle. Also, it should be noted that Table 2 only represents a few of the vast number of AQ polymer blends (i.e. various ratios of different AQ polymers) that one may potentially prepare.

TABLE 1
AQ Polymer Powder Examples

| Example | AQ Polymer | Appearance |
|---|---|---|
| 1 | AQ29 | White Powder/Solid |
| 2 | AQ38 | White Powder/Solid |
| 3 | AQ48 | White Powder/Solid |
| 4 | AQ55 | White Powder/Solid |
| 5 | Eastek 1200 | White Powder/Solid |
| 6 | AQ1045 | Clear Sticky Solid |
| 7 | AQ1350 | White Powder** |
| 8 | AQ1950 | White Powder** |
| 9 | AQ2150 | White Powder** |
| 10 | AQ2350 | White Powder** |
| 11 | EASTONE | White Powder/Solid |

**= Over time, the initial powder becomes a fused sticky mass.

Example of Dispersion Preparation from AQ Powder:

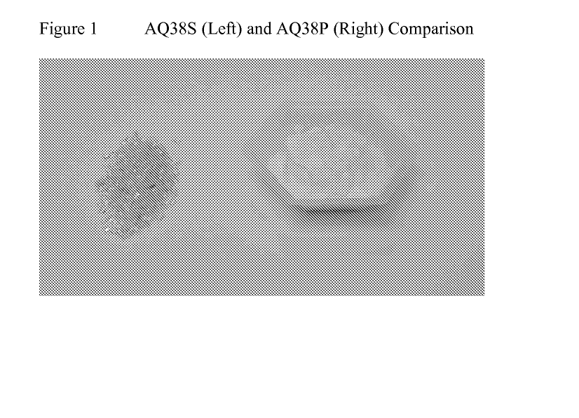

Using these polymer powder forms, one may readily prepare dispersions. The following represents an example to prepare a powder (or powder blend) dispersion. It will be understood that this example has been included merely for purpose of illustration and not intended to limit the scope of the invention (e.g. powder grinding to control particle size distribution, the usage of different solvents (water, ethanol, etc.), temperatures, and mixing procedures) unless otherwise specifically indicated. A 7.3% dispersion preparation has been included: AQ38S pellets (3.0 g) and AQ38 powder (AQ38P; 3.0 g) were weighed out (FIG. 1) into separate beakers (50 mL), both contained a stir-bar. At the beginning of the preparation (time=0) and at the same time, the stirrers were placed on the same stirring setting. Next, room temperature water (38 mL) was added to each of the beakers. After 5-10 min, the majority of the powder form was completely dispersed; whereas the pellets, at 5-10 min of mixing, had very limited dispersion formation.

TABLE 2
AQ Polymer Powder Blend Examples

| Example | Polymer Blend | Appearance |
|---|---|---|
| 1 | AQ29:AQ1045 (5:1 Blend) | White Powder/Solid |
| 2 | AQ29:AQ1045 (4:1 Blend) | White Powder/Solid |
| 3 | AQ29:AQ1350 (5:1 Blend) | White Powder/Solid |
| 4 | AQ29:AQ1350 (4:1 Blend) | White Powder/Solid |
| 5 | AQ29:AQ1950 (5:1 Blend) | White Powder/Solid |
| 6 | AQ29:AQ1950 (4:1 Blend) | White Powder/Solid |
| 7 | AQ29:AQ2150 (4:1 Blend) | White Powder/Solid |
| 8 | AQ29:AQ2350 (4:1 Blend) | White Powder/Solid |
| 9 | AQ29:EASTONE (1:1 Blend) | White Powder/Solid |
| 10 | AQ38:AQ1045 (5:1 Blend) | White Powder/Solid |
| 11 | AQ38:AQ1045 (4:1 Blend) | White Powder/Solid |
| 12 | AQ38:AQ1350 (5:1 Blend) | White Powder/Solid |
| 13 | AQ38:AQ1350 (4:1 Blend) | White Powder/Solid |
| 14 | AQ38:AQ1950 (5:1 Blend) | White Powder/Solid |
| 15 | AQ38:AQ1950 (4:1 Blend) | White Powder/Solid |
| 16 | AQ38:AQ2150 (4:1 Blend) | White Powder/Solid |
| 17 | AQ38:AQ2350 (4:1 Blend) | White Powder/Solid |
| 18 | AQ48:AQ1045 (5:1 Blend) | White Powder/Solid |
| 19 | AQ48:AQ1045 (4:1 Blend) | White Powder/Solid |
| 20 | AQ48:AQ1350 (5:1 Blend) | White Powder/Solid |
| 21 | AQ48:AQ1350 (4:1 Blend) | White Powder/Solid |
| 22 | AQ48:AQ1950 (5:1 Blend) | White Powder/Solid |
| 23 | AQ48:AQ1950 (4:1 Blend) | White Powder/Solid |
| 24 | AQ48:AQ2150 (4:1 Blend) | White Powder/Solid |
| 25 | AQ48:AQ2350 (4:1 Blend) | White Powder/Solid |
| 26 | AQ55:AQ1045 (5:1 Blend) | White Powder/Solid |
| 27 | AQ55:AQ1045 (4:1 Blend) | White Powder/Solid |
| 28 | AQ55:AQ1350 (5:1 Blend) | White Powder/Solid |
| 29 | AQ55:AQ1350 (4:1 Blend) | White Powder/Solid |
| 30 | AQ55:AQ1950 (5:1 Blend) | White Powder/Solid |
| 31 | AQ55:AQ1950 (4:1 Blend) | White Powder/Solid |
| 32 | AQ55:AQ2150 (4:1 Blend) | White Powder/Solid |
| 33 | AQ55:AQ2350 (4:1 Blend) | White Powder/Solid |
| 34 | Eastek 1200:AQ1045 (4:1 Blend) | White Powder/Solid |
| 35 | Eastek 1200:AQ1350 (4:1 Blend) | White Powder/Solid |
| 36 | Eastek 1200:AQ1950 (4:1 Blend) | White Powder/Solid |
| 37 | Eastek 1200:AQ2150 (4:1 Blend) | White Powder/Solid |
| 38 | Eastek 1200:AQ2350 (4:1 Blend) | White Powder/Solid |
| 39 | EASTONE:AQ1045 (5:1 Blend) | White Powder/Solid |
| 40 | EASTONE:AQ1045 (4:1 Blend) | White Powder/Solid |
| 41 | EASTONE:AQ1350 (5:1 Blend) | White Powder/Solid |
| 42 | EASTONE:AQ1350 (4:1 Blend) | White Powder/Solid |
| 43 | EASTONE:AQ1950 (5:1 Blend) | White Powder/Solid |
| 44 | EASTONE:AQ1950 (4:1 Blend) | White Powder/Solid |
| 45 | EASTONE:AQ2150 (4:1 Blend) | White Powder/Solid |
| 46 | EASTONE:AQ2350 (4:1 Blend) | White Powder/Solid |
| 47 | AQ38:AQ55:AQ1045 (2:2:1 Blend) | White Powder/Solid |

Examples of AQ Powders Containing Carriers and/or Actives:

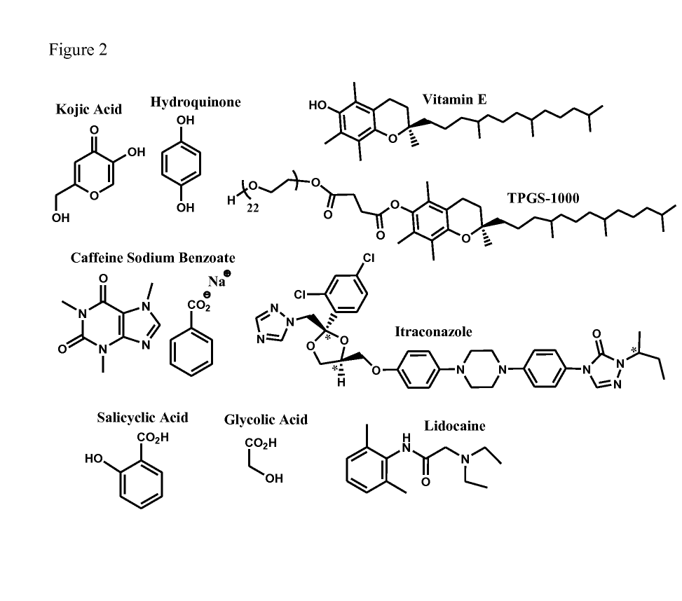

This invention can be further illustrated by the following examples (summarized in FIG. 2 and Table 3), although it will be understood that these examples are included merely for purposes of illustration and are not intended to limit the scope of the invention unless otherwise specifically indicated.

General Procedure:

A chosen sulfo-polyester polymer dispersion and/or dispersion blend is mixed while the desired carriers (e.g. TPGS 1000) and/or actives (e.g. kojic acid, hydroquinone, vitamin E, lidocaine, itraconazole, caffeine sodium benzoate, salicyclic acid, glycol acid) are added; depending on the desired properties, carriers and/or actives may be added as solids, and/or as suspensions, and/or as solutions. The corresponding dispersions are frozen and lyophilized to afford powders and/or solids; all methodology includes the act of bringing the carriers and/or active ingredient(s) into association with the one or more sulfo-polyester polymers. Therefore, compositions (formulations) are prepared by blending active ingredient(s) with a liquid carrier or a finely divided solid carrier, and/or both, and then, if needed, shaping the product into a desired formulation (e.g. powder grinding to control particle size distribution, the usage of different solvents (water, ethanol, etc.) and their temperatures and mixing procedures).

TABLE 3
AQ Polymer Powders + Carriers and/or Actives

| Example | AQ Polymer + Carrier/Active |
|---|---|
| 1 | AQ38 + Vitamin E (1.5% Loading) |
| 2 | AQ55 + Kojic acid (5% Loading) |
| 3 | AQ55 + Hydroquinone (11% Loading) |
| 4 | AQ55 + Hydroquinone (4% Loading) |
| 5 | AQ55 + Glycolic Acid (40% Loading) |
| 6 | AQ55 + Salicyclic Acid (4.5% Loading) |
| 7 | AQ55 + TPGS1000 (11% Loading) |
| 8 | AQ55 + Caffeine-Na-Benzoate (3.6% Loading) |
| 9 | AQ55 + Itraconazole (2% Loading) |
| 10 | AQ55 + Lidocaine (0.9% Loading) |

### Example of AQ Powder Plus an Active Increasing Bioavailability:

Methods to monitor itraconazole and hydroxy-itraconazole via oral dosing have been previously described (Buchanan et al, J. Pharm. Sci. 96 (11) 2007). General procedures used for the follow studies were as follows: Male (234-252 g) Sprague-Dawley rats were purchased from Harlan (Indianapolis, Ind., USA). For each dose group, three animals were used to investigate itraconazole pharmacokinetics. Animals were housed in groups of three at 22.2±1.1° C. and 55±15% humidity with 12 h dark light cycles. Dosing occurred 2.0-2.5 h after the beginning of a light cycle. All animals had free access to water and were fasted 15-16 h prior to dosing; food was returned 5 h post-dose.

Itraconazole was purchased from Apin Chemicals Ltd (Abingdon, Oxon, UK). Itraconazole (200 mg) was weighed out and the particle size was reduced manually using a mortar and pestle. Subsequently, two different formulations were prepared as follows: i) itraconazole (30 mg) was transferred to a glass vial and 170 mg of carboxymethyl cellulose (CMC; microgranular, 25-60 μm) was added; and ii) itraconazole (30 mg) was transferred to a glass vial and 170 mg of AQ55 powder was added. The vials were capped and placed onto a mechanical roller (1 h). To help remove clumps and/or aggregates that might have formed while mixing, the materials were individually removed and passed through a 35 mesh sieve screen. After additional mixing via the mechanical roller (45 min), the resulting formulations were used to prepare dosing capsules. Using a filling funnel, the formulations were encapsulated into hard shell Torpac Lock ring gel (size 0, in vitro dissolution; and size 9, in vivo dosing) capsules (Torpac, USA).

Animals were dosed orally using a Torpac capsule syringe (Torpac, USA); following the capsule dosing, 0.5 mL of water was orally administered to facilitate capsule movement to the stomach. Using tail-vein collection (i.e. the distal portion was transected, 2-3 mm), blood samples (125 μL) were collected using mini-capillary blood collection tubes that contained EDTA di-potassium salt (SAFE-T-FILL®; RAM Scientific Inc., Yonkers, N.Y., USA). Immediately after filling the individual samples, the tubes were capped, mixed, stored on dry ice and kept frozen (−80±10° C.) until sample preparation and subsequent LC/MS/MS analysis.

The samples were assayed using a Sciex 4000-QTrap mass spectrometer (Applied Biosystems, Foster City, Calif., USA) equipped with a Shimadzu HPLC, a PEAK Scientific API Systems gas generator (Bedford, Mass., USA) and Leap auto-sampler (Carrboro, N.C.). Analyst 1.4.1 was used for data acquisition. Prism 4.02™ (GraphPad Software, Inc.; San Diego, Calif.) was used for data analysis, graphing, and statistical analysis. Ten microliters of the extracted samples were injected onto a Zorbax extended-C18 50×4.6 mm, 5-micron 80 Å column (Agilent Technologies, UK). The column temperature was set at 40±1° C. using a Temperature Control Module (Analytical Sales and Services; Pompton Plains, N.J.). A binary gradient was used; solvent A was a 10 mM ammonium acetate solution containing 0.1% formic acid and solvent B was a 50:50 mixture of methanol:acetonitrile. Using a flow-rate of 0.4 mL/min, the following gradient was used for the HPLC separations: 95% A for 1.0 min; brought to 5% A at 3.0 min and held for 3.5 min; returned to 95% A after 0.5 min and held at 95% A for 1.5 min (8.5 min total).

An extraction solution containing acetonitrile:methanol (1:1) with 0.2% HClaq was used. In individual animal sets, the blood samples were removed from the freezer (−80±10° C.) and allowed to warm to ambient temperature (5-10 min). The tubes were vortexed (3-5 sec) and extraction solution (250 μL) was added, vortex mixed (3-5 sec), and sonicated (10 min, Branson 2510 Ultrasonic cleaner; Danbury, Conn., USA). The tubes were centrifuged at 13,200 rpm (10 min) using an Eppendorf minispin centrifuge (Hamburg, Germany). The supernatant was transferred into individual wells of a 96-well plate. The 96-well plate was sealed and centrifuged at 3000 rpm for 10 min at 10° C. (Labofuge 400R Centrifuge; VWR, West Chester, Pa., USA). The 96-well plate was placed into the LEAP auto-sampler cool-stack (6.0±0.1° C.) and analyzed via LC/MS/MS.

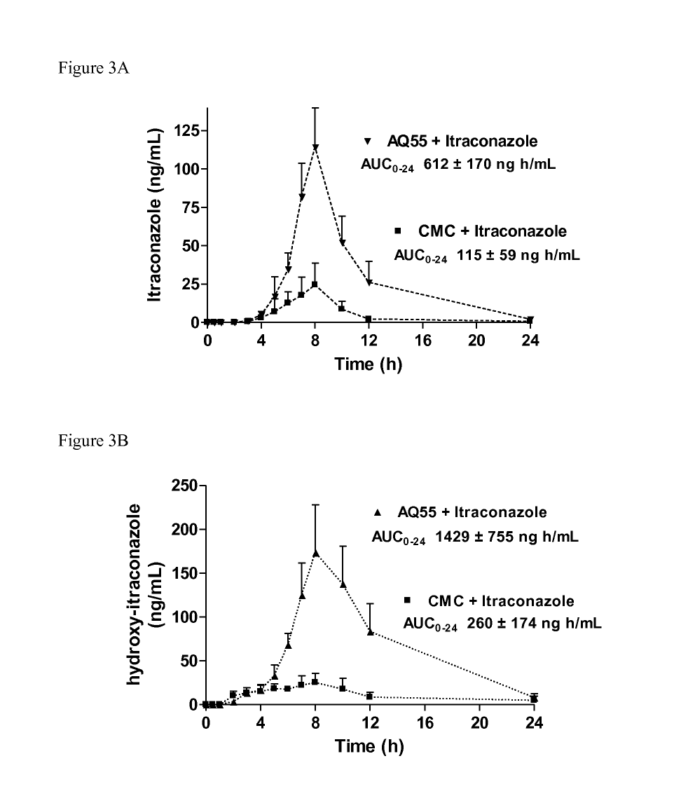

The oral dose blood concentration-time data (n=3; ±SD) for itraconazole and hydroxy-itraconazole for the two dosing groups are presented in FIG. 3A and FIG. 3B, respectively. A visual inspection of these data reveals that the Tₘₐₓ was ˜8 h. These data illustrate that the presence of AQ55 powder in an oral dose increased the overall amount of itraconazole absorbed; an average AUC₀₋₂₄ₕ increase from 115 ng h/mL to 612 ng h/mL. An increase in the AUC₀₋₂₄ₕ equates to an increase in the oral bioavailability. Furthermore, hydoxy-itraconazole—an active metabolite—was also found in higher concentrations with animals dosed with the AQ powder.

### CONCLUSION

Sulfo-polyester polymer dispersions and dispersion blends in the presence of carriers and/or actives were prepared. Samples were frozen and lyophilized to afford white to off-white solids; the majority of these solids were not tacky, easy to handle, and dispersible in water and/or an appropriate solvent system. In addition to the ability that one may readily prepare novel Sulfo-polyester polymer powder blends which may be used to readily prepare dispersions, this methodology significantly removed residual volatile components that were present (i.e. there are sweet smelling volatiles present) in the original dispersions. The volatile components present may not be desired for certain end-uses. The ability to offer powder sulfopolyester and/or powder sulfoployester blends with and without other materials present (i.e. surfactants and/or actives) may offer users the advantage of easily and quickly preparing dispersions with room temperature or warm water, or solvent mixtures, with a faster dissolution rate.

The invention has been described in detail with particular reference to preferred embodiments thereof, but it will be understood that variations and modifications can be effected within the spirit and scope of the invention.

We claim:

1. A composition comprising at least one sulfonated copolyester and at least one active agent, wherein said composition is a powder.

2. The composition according to claim 1, wherein the active agent is selected from the group consisting of a pharmaceutical agent, a veterinary agent, an agricultural agent, a flavor agent, a fragrance, a cosmetic agent, and mixtures thereof.

3. The composition according to claim 1, wherein said active agent is present in an amount of up to about 90% by weight.

4. The composition according to claim 3, wherein said active agent is present in an amount of from about 40% to about 0.5% by weight.

5. The composition according to claim 4, wherein said active agent is present in an amount of from about 15% to about 1.0% by weight.

6. The composition according to claim 1, wherein the composition further comprises at least one carrier.

7. The composition according to claims 6, wherein the carrier is selected from the group consisting of solvents, diluents, binders, lubricants, absorbents, colorants, fragrances, sweeteners, disintegrants, and mixtures thereof.

8. A method of increasing the bioavailability of an active agent comprising administering the composition of claim 1 to a subject.

9. The method of claim 8, wherein the subject is a mammal.

10. The method of claim 8, wherein the subject is a human.

11. The method of claim 8, wherein the subject is a plant, shrub, or foliage.

12. A method for producing a sulfonated copolyester powder containing an active agent comprising:
a) dispersing at least one sulfonated copolyester and at least one active agent in a solvent to form a dispersion; and
b) drying said dispersion to form a powder.

13. The method according to claim 12, further comprising freezing said dispersion prior to drying.

14. The method according to claim 12, wherein said drying is by lyophilization, spray-drying, jet milling, spray freeze-drying, fluidized-bed spray coating, or supercritical fluid methods.

15. The method according to claim 12, further comprising dispersing a carrier in said solvent.

16. A method for producing a sulfonated copolyester powder containing an active agent comprising:
a) dispersing at least one sulfonated copolyester in a solvent to form a dispersion; and
b) drying said dispersion to form a powder; and
c) mixing (grinding, milling, and the like) said sulfonated copolyester powder with at least one active agent.

17. The method according to claim 16, further comprising freezing said dispersion prior to drying.

18. The method according to claim 16, wherein said drying is by lyophilization, spray-drying, jet milling, spray freeze-drying, fluidized-bed spray coating, or supercritical fluid methods.

19. A cosmetic composition, comprising the composition according to claim 1 and a cosmetically acceptable carrier.

20. A pharmaceutical composition, comprising the composition according to claim 1 and a pharmaceutically acceptable carrier.

21. A veterinary composition, comprising the composition according to claim 1 and a veterinary acceptable carrier.

22. An agricultural composition, comprising the composition according to claim 1 and an agriculturally acceptable carrier.
